(19) United States
(12) Patent Application Publication
(10) Pub. No.: US 2024/0379227 A1
(43) Pub. Date: Nov. 14, 2024

(54) CONSTRUCTION OF NEAREST NEIGHBOR STRUCTURES FOR GRAPH MACHINE LEARNING TECHNOLOGIES

(71) Applicant: NEC Laboratories Europe GmbH, Heidelberg (DE)

(72) Inventor: Federico Errica, Heidelberg (DE)

(21) Appl. No.: 18/347,598

(22) Filed: Jul. 6, 2023

Related U.S. Application Data
(60) Provisional application No. 63/500,936, filed on May 9, 2023.

(51) Int. Cl. G16H 50/20; G16H 20/10

(52) U.S. Cl. CPC G16H 50/20; G16H 20/10

(57) ABSTRACT

A method for construction of nearest neighbor structures includes determining a set of cross-class neighborhood similarities based on a set of distributions of data obtained by applying a model to data present in a dataset. The method selects a first cross-class neighborhood similarity from the set of cross-class neighborhood similarities based on one or more inter-class cross-class neighborhood similarities and one or more intra-class cross-class neighborhood similarities, and builds a nearest neighbor graph based on the first cross-class neighborhood similarity. The present invention can be used in a variety of applications including, but not limited to, several anticipated use cases in drug development, material synthesis, and medical/healthcare.

### CROSS-REFERENCE TO PRIOR APPLICATION

Priority is claimed to U.S. Provisional Application No. 63/500,936, filed on May 9, 2023, the entire contents of which is hereby incorporated by reference herein.

### FIELD

The present invention relates to Artificial Intelligence (AI) and machine learning (ML), and in particular to a method, system and computer-readable medium for constructing nearest neighbor structures in a graph machine learning framework for use in one or more machine learning tasks.

### BACKGROUND

Graph machine learning (GML) technology operates on data represented as a graph of a set of connected entities. When working on classification problems for tabular data (e.g., when no connectivity is available), researchers have tried to apply GML methods on top of artificial structures (see Malone, Brandon; Garcia-Duran, Alberto; and Niepert, Mathias, “Learning representations of missing data for predicting patient outcomes,” Workshop on Deep Learning on Graphs: Method and Applications (2021), which is hereby incorporated by reference herein). Most of these artificial structures exploit the similarity between entities' attributes to create connections between them, but it is not clear when they are useful or not. Recently, however, the cross-class neighborhood similarity (CCNS) has been proposed as a tool to understand when the structure can be useful or not for entity classification in a graph (see Ma, Yao; Liu, Xiaorui; Shah, Neil; and Tang, Jiliang, “Is homophily a necessity for graph neural networks?” arXiv: 2106.06134 (2021), which is hereby incorporated by reference herein).

### SUMMARY

In an embodiment, the present invention provides a method for construction of nearest neighbor structures. The method includes determining a set of cross-class neighborhood similarities based on a set of distributions of data obtained by applying a model to data present in a dataset. The method selects a first cross-class neighborhood similarity from the set of cross-class neighborhood similarities based on one or more inter-class cross-class neighborhood similarities and one or more intra-class cross-class neighborhood similarities, and builds a nearest neighbor graph based on the first cross-class neighborhood similarity.

The present invention can be used in a variety of applications including, but not limited to, several anticipated use cases in drug development, material synthesis, and medical/healthcare.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 schematically illustrates a system for constructing a nearest neighbor graph and using it for training a graph machine learning framework according to an embodiment of the present invention;

FIG. 2 schematically illustrates a pipeline for constructing a nearest neighbor graph and using it for training a graph machine learning framework to solve a graph machine learning task according to an embodiment of the present invention;

FIG. 3A illustrates assumptions on the data distribution through a graphical model (a hierarchical mixture of distributions), where observed variable X is shaded and latent variables C and N are white circles;

FIG. 3B illustrates a visual example of the data distribution generated by the graphical model, with an intuitive visualization of the class posterior mass around a point xᵤ;

FIG. 4 illustrates a table with a node classification mean and standard deviation results for a structure-agnostic baseline (for example, a multi-layer perceptron (MLP)) compared to graph machine learning methods applied to artificially built k-NN graphs;

FIG. 5A illustrates a graph in which each curve specifies the variation of squared error distance (SED) against different values of k for different hyper parameters' configurations;

FIG. 5B illustrates in (a) the CCNS lower bound, in (b) its Monte Carlo approximation for ε≈0.1, obtained without creating any graph, and in (c) the true cross-class neighborhood similarity for the example of FIGS. 2A and 2B, and a 5-NN graph;

FIG. 6 is a block diagram of an exemplary processing system, which can be configured to perform any and all operations disclosed herein.

FIG. 7 illustrates the main statistics of the used datasets;

FIG. 8 illustrates the hyper-parameters tried during model selection for all used benchmarks;

FIG. 9 illustrates the hyper-parameters tried for the qualitative experiments;

FIG. 10 illustrates histograms of the learned k and ε for the different configurations of FIG. 9;

FIGS. 11A-11E illustrates a comparison of the true cross-class neighborhood similarity for varying values of k and the Monte Carlo approximation for a random initialization of a 5-class data model;

FIGS. 12-14 illustrate histograms of the k, number of layers, or number of hidden units, chosen by each model on the different datasets during a 10-fold cross validation for risk assessment; and

FIGS. 15-17 illustrate an ablation study for the k, number of layers, or number of hidden units, chosen by each model on the different datasets.

### DETAILED DESCRIPTION

Embodiments of the present invention provide a theoretically grounded pipeline to efficiently assess the quality of nearest neighbor artificial graphs for the classification of tabular data using graph representation learning methodologies. For example, embodiments of the present invention can be applied to improve graph machine learning technologies by enabling computation of more accurate graphs, and thereby providing for more accurate predictions and decisions in applications of graph machine learning tasks, while at the same time reducing the computational burden and saving computational resources and memory in the graph machine learning frameworks. For instance, previously, to use the cross-class neighborhood similarity (CCNS) with artificial graphs, one has to: 1) choose a structure construction method; 2) compute the structure for a user-specified parametrization of the chosen method (for example, the parameter k in a k-nearest neighbor (kNN) algorithm); and 3) compute the cross-class neighborhood similarity on the available data. Finding the best parametrization can be computationally and memory intensive if the number of samples to connect is high, because it might involve the re-computation of many graphs.

In some instances, embodiments of the present invention solve this technical problem and reduce the computational burden by using a method that builds nearest neighbor structures (e.g., k-nearest neighbors (kNN) or epsilon-radius structures). Using a theoretical approximation of the cross-class neighborhood similarity, embodiments of the present invention can determine (e.g., estimate) if a nearest neighbor structure can provide a satisfactory cross-class neighborhood similarity without building the graphs. According to one or more embodiments, a simple graphical model can be used to obtain an approximation of the entities' attributes data distributions.

According to a first aspect, the present disclosure provides a method for construction of nearest neighbor structures. The method includes determining a set of cross-class neighborhood similarities based on a set of distributions of data obtained by applying a model to data present in a dataset. The method selects a first cross-class neighborhood similarity from the set of cross-class neighborhood similarities based on one or more inter-class cross-class neighborhood similarities and one or more intra-class cross-class neighborhood similarities, and builds a nearest neighbor graph based on the first cross-class neighborhood similarity.

According to a second aspect, the method according to the first aspect further comprises applying the model to data present in the dataset by receiving data for the dataset in a tabular from via user input, and modeling each data point of the data that belongs to a class (C) as a mixture of Gaussian distributions (M).

According to a third aspect, the method according to the first or the second aspect further comprises applying the model to data present in the dataset by determining learned parameters for the set of distributions of data present in the dataset.

According to a fourth aspect, the method according to any of the first to the third aspects further comprises determining the set of cross-class neighborhood similarities by using the learned parameters to compute a value of the cross-class neighborhood similarities, wherein the nearest neighbor graph is built based on the value of the cross-class neighborhood similarities.

According to a fifth aspect, the method according to any of the first to the fourth aspects further comprises that the value of the cross-class neighborhood similarities is computed using Monte Carlo simulations.

According to a sixth aspect, the method according to any of the first to the fifth aspects further comprises training a graph machine learning model based on the nearest neighbor graph, and performing predictive tasks using the trained graph machine learning model.

According to a seventh aspect, the method according to any of the first to the sixth aspects further comprises that the model applied to the data present in the dataset is a Hierarchical Naïve Bayes model.

According to an eighth aspect, the method according to any of the first to the seventh aspects further comprises determining the set of distributions by computing a probability that a first node, belonging to a first class, has a nearest neighbor node, belonging to a second class.

According to a ninth aspect, the method according to any of the first to the eighth aspects further comprises selecting the first cross-class neighborhood similarity by determining a trade-off between the one or more inter-class cross-class neighborhood similarities and the one or more intra-class cross-class neighborhood similarities.

According to a tenth aspect, the method according to any of the first to the ninth aspects further comprises that the nearest neighbor graph includes a selected node at a center of hypercube, and a set of neighbors of the selected node within the hypercube, wherein the hypercube is formed based on the first parameter.

According to an eleventh aspect, the method according to any of the first to the tenth aspects further comprises that the data present in the dataset comprises electronic health records corresponding to a plurality of patients, wherein the electronic health records comprise heart rate, oxygen saturation, weight, height, glucose, temperature associated with each patient in the plurality of patients, the nearest neighbor graph is built based on the electronic health records present in the dataset, a graph machine learning model is trained using the nearest neighbor graph, and a clinical risk is predicted for a patient using the trained graph machine learning model.

According to a twelfth aspect, the method according to any of the first to the eleventh aspects further comprises that the data present in the dataset comprises genomic activity information corresponding to a plurality of patients, wherein the genomic activity of each patient identifies a response of the respective patient to a drug, the nearest neighbor graph is built based on the genomic activity information present in the dataset, a graph machine learning model is trained using the nearest neighbor graph, and a suitability of a patient for a drug trial is predicted using the graph machine learning model.

According to a thirteenth aspect, the method according to any one of the first to the twelfth aspects further comprises that the data present in the dataset comprises soil data corresponding to a plurality of areas, wherein the soil data comprises humidity, temperature, and performance metrics related to different areas, the nearest neighbor graph is built based on the soil data present in the dataset, a graph machine learning model is trained using the nearest neighbor graph, and a quality of an input soil type is predicted based on the nearest neighbor graph.

A fourteenth aspect of the present disclosure provides a computer system programmed for performing automated sharing of data and analytics across a data space platform, the computer system comprising one or more hardware processors which, alone or in combination, are configured to provide for execution of the following steps: determining a set of cross-class neighborhood similarities based on a set of distributions of data obtained by applying a model to data present in a dataset; selecting a first cross-class neighborhood similarity from the set of cross-class neighborhood similarities based on one or more inter-class cross-class neighborhood similarities and one or more intra-class cross-class neighborhood similarities; and building a nearest neighbor graph based on the first cross-class neighborhood similarity.

A fifteenth aspect of the present disclosure provides a tangible, non-transitory computer-readable medium for performing automated sharing of data and analytics across a data space platform having instructions thereon, which, upon being executed by one or more processors, provides for execution of the following steps: determining a set of cross-class neighborhood similarities based on a set of distributions of data obtained by applying a model to data present in a dataset; selecting a first cross-class neighborhood similarity from the set of cross-class neighborhood similarities based on one or more inter-class cross-class neighborhood similarities and one or more intra-class cross-class neighborhood similarities; and building a nearest neighbor graph based on the first cross-class neighborhood similarity.

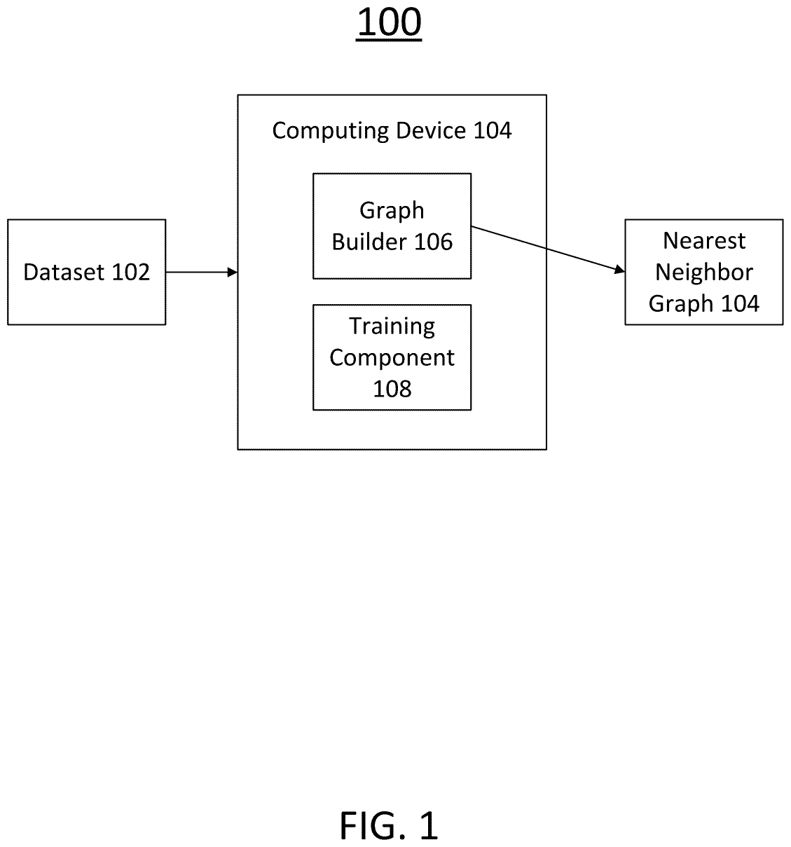

FIG. 1 depicts a schematic illustration of a system for constructing a nearest neighbor graph and using it for training a graph machine learning framework according to an embodiment of the present invention. System 100 of FIG. 1 depicts a tabular dataset 102 that includes a tabular dataset that includes numerical features. In some embodiments, the data of the tabular dataset 102 can be provided by a user. Computing device 104 of system 100 can be configured to instruct and/or use a graph builder 106 to approximate the data distribution of the dataset 102 using one or more graphical models. In some embodiments, the graphical model can be selected by a user. In some other embodiments, the computing device selects the specific graphic model from a plurality of graphical models As the data from the dataset 102 is fit in a model by the graph builder 106, the computing device 104 can also store learned parameters of the following distributions in the dataset 102: the prior class distribution p(C=c); the mixture weights distribution p(M=m|C=c); and the emission distribution p(X=x|M=m,C=c). The computing device 104 can also instruct graph builder 106 to determine the best theoretical CCNS distance using the learned parameters. For example, the learned parameters are used to compute a theoretical approximation of the cross-class neighborhood similarity under the nearest neighbor graph to look for “good” cross-class neighborhood similarity, which will be described in further detail below. The computing device 104 can provide instructions to the graph builder 106 to build a nearest neighbor graph based on the best theoretical cross-class neighborhood similarity distance. Then, the computing device 104 can instruct the training component 108 of system 100 to train a graph machine learning method using the nearest neighbor graph.

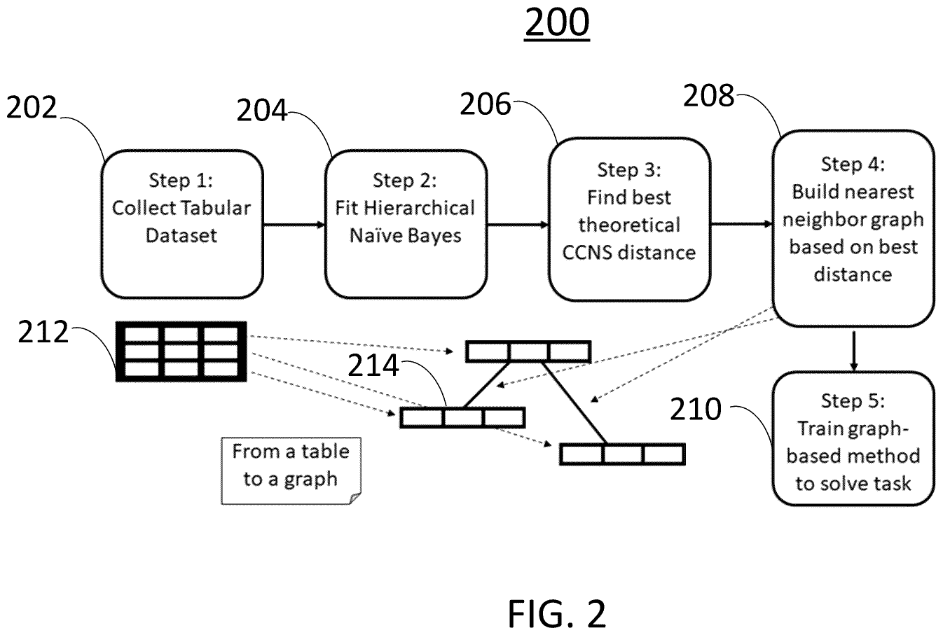

FIG. 2 schematically illustrates a pipeline for constructing a nearest neighbor graph and using it for training a graph machine learning framework to solve a graph machine learning task according to an embodiment of the present invention. For instance, FIG. 2 shows a method 200 including a series of intermediate steps 202-210 to transform a tabular dataset (e.g., the tabular dataset 212) into a graph of entities (e.g., the graph of entities 214) to classify. For instance, an embodiment of the present invention provides a module (e.g., a processor and/or controller) that approximates the cross-class neighborhood similarity without explicitly computing it in step 206 of the method.

Existing technology for computing cross-class similarity require a connectivity structure between the various entities of a dataset. Thus, prior methods simply compute the cross-class neighborhood similarities using the edges of the existing connectivity structure. In the absence of a graph, a possible method to compute cross-class neighborhood similarities would be to try many different connectivities according to some criteria, evaluate the cross-class neighborhood similarities each time, and then pick the one with the best cross-class neighborhood similarities. However, both of these methods are computationally intensive.

In contrast, embodiments of the present invention need not build the graphs in advance, and enable to conserve computational resources, compute time and/or compute power. The cross-class neighborhood similarity can be approximated by estimating a theoretical distance between the neighbors associated with the most promising nearest neighbor graph, and hence there is no need to iteratively build nearest neighbor graphs each time to evaluate the cross-class neighborhood similarities.

In some embodiments, the cross-class neighborhood similarity computes a similarity score between each pair of classes in the task, by comparing the average similarity in class label neighboring distributions between all nodes of the different classes. For example, a first node belonging to class 0 has 3 neighbors. Each of the neighbors of the node belong to classes 0, 1, 2, respectively. Similarly, a second node belonging to class 1 has 4 neighbors, each of which belong to classes 0, 0, 0, and 1 respectively. The empirical class histograms between the first node of class 0 and the second node of class 1 will look very different, hence the similarity between these two nodes will be low. The CCNS computes this similarity for all nodes of classes 0 and 1. First, the CCNS computes an aggregated histogram of neighboring classes for all nodes of class 0, and then for all nodes of class 1. Therefore, CCNS computes the similarity (using cosine similarity for instance) between the two histograms. The process is repeated for all pairs of classes.

The computation of CCNS uses the graph connectivity that is already available and applies the definition of CCNS. But this process is resource intensive. Instead, the CCNS distance is approximated. A first step of the approximation is to estimate, using theoretical arguments, the most promising distance to have in a nearest neighbor graph to maximize the CCNS, in case an approximation of the distributions of the node features for each separate class are known. Then, the nearest neighbors graph is created that satisfies that maximum distance between neighbors.

In some embodiments in step 202, a tabular dataset with numerical features can be provided by a user. For instance, the computing device 104 can obtain (e.g., receive) user input indicating and/or including the tabular dataset (e.g., the tabular dataset 212).

In step 204, the method 200 includes fitting a Hierarchical Naïve Bayes (HNB) model. For instance, given the tabular dataset 212, the next step is to approximate the data distribution using a graphical model (e.g., a specific graphical model), such as a Hierarchical Naïve Bayes (see, e.g., Langseth, Helge; and Nielsen, Thomas D., “Classification using hierarchical naive Bayes models,” Machine learning 63:135-159 (2006), which is hereby incorporated by reference herein). In some embodiments, each data point that is a sample/row in the table, of a class (e.g., specific class c) can be modeled as a mixture of M Gaussian distributions.

For example, the method 200 starts with the computing device 104 accessing the dataset 102 (e.g., the tabular dataset 212). For instance, the computing device 104 obtains, from a user, user input indicating the dataset. The computing device 104 can provide data from the dataset 102 to the graph builder 106. Graph builder 106 can approximate the data distribution of the dataset 102 using a graphical model (e.g., HNB model). For example, the computing device 104 can fit data from the dataset 102 in the HNB model (e.g., each data point from a sample/row in the table 212 of a class (e.g., specific class c) can be modeled as a mixture of M Gaussian distributions).

Background to understand the following mathematical results are described below. For instance, a graph g of size N is a quadruple ( [symbol] , ξ, X, [symbol] ), where [symbol] ={1, . . . , N} represents the set of nodes and ξ is the set of directed edges (u, v) from node u to node v. The symbol X={Xᵤ, ∀ u∈ [symbol] } defines the set of independent and identically distributed (i.i.d) random variables with realizations {xᵤ∈R^{D}, ∀u∈ [symbol] }. The same definition applies to the set of target variables [symbol] ={Yᵤ, ∀u∈ [symbol] )} and their realizations {yᵤ∈|C|, ∀u∈ [symbol] }. The symbol [symbol] _{c} denotes the subset of nodes with target label c∈|C|, and the neighborhood of node u is defined as [symbol] ={v∈ [symbol] |(v,u)∈ξ}.

A Gaussian (or normal) univariate distribution with mean and variance μ, σ²∈R is represented as [symbol] (·;μ, σ²), using μ∈R^{D}, Σ∈R^{D×D} for the multivariate case. The probability density function (p.d.f.) of a univariate normal random variables parametrized by μ, σ is denoted by ϕ(·) together with its cumulative distribution function (c.d.f.) F(w)=

$$Φ ( \frac{w - μ}{σ} ) = \frac{1}{2} ( 1 + erf ( \frac{w - μ}{σ \sqrt{2}} ) ) ,$$

related to a specific random variable.

Embodiments of the present invention (e.g., the computing device 104) transform the initial task into a node classification problem, where the different samples are the nodes of a single graph, and the edges are computed by a nearest-neighbor algorithm. In some instances, the true data distribution p(X=x) is defined by the HNB model, with the |C| latent classes modeled by a categorical random variable C with prior distribution p(C=c) and |M| mixtures for each class modeled by M˜p(M=m|C=c). In some examples, it is further provided that the attributes are conditionally independent when the class c and the mixture m are known, such as p(X=x)=Σ_{ci}^{|C|}p(c)Σₘ₌₁^{|M|}p(m|c)Π_{f=1}^{D}p(x_{f}|m,c). This graphical model allows consideration of continuous attributes and, to some extent, categorical values. Hereinafter, p(X_{f}=x_{f}|m,c)= [symbol] (x_{f};μ_{cmf}, σ_{cmf}²) is obtained, which for notational convenience can be written as p(x|m,c)= [symbol] (x;μ_{mc},Δ_{mc}) with diagonal covariance Δ_{mc}=diag(σ_{mc}_{₁}²,σ_{mc}_{_{D}}²). Finally, it is assumed that the neighboring class labels of a node are independent from each other.

In some embodiments, the transformation into a node classification problem is simply a byproduct of the graph creation process. For example, the initial task is a standard classification, in which each data element of the dataset 102 has independent features and is processed in isolation. Additionally, connecting samples using an artificial structure effectively creates a graph where each node is one of the samples, and each sample still needs to be classified. The theoretical results that are provided rely on the assumption that the data distribution is specified by a Hierarchical Naïve Bayes model, which models the distribution of the data as a mixture of Gaussian distributions, where the mixing weights of each mixture component are obtained by another mixture of categorical distributions.

To refer to the surroundings of a point in space, the notion of hypercubes (e.g., an n-dimensional analogue of a square and a cube) is used. A hypercube of dimension D centered at point x∈R^{D} of side length c is the set of points given by the Cartesian product

$${H}_{ε} ( x ) = [ {x}_{1} - \frac{ε}{2} , {x}_{1} + \frac{ε}{2} ] \times … \times [ {x}_{D} - \frac{ε}{2} , {x}_{D} + \frac{ε}{2} ] .$$

For example, using the nearest neighbor algorithm, the computing device 104 can determine neighboring nodes corresponding to a selected node using the concept of hypercubes. The selected node is placed at the center of a hypercube of dimension D. The neighbors of the selected node placed at the center of the hypercube are determined by the Cartesian product

$${H}_{ε} ( x ) = [ {x}_{1} - \frac{ε}{2} , {x}_{1} + \frac{ε}{2} ] \times … \times [ {x}_{D} - \frac{ε}{2} , {x}_{D} + \frac{ε}{2} ] ,$$

where E is a side length of the hypercube and x∈R^{D} depicts the selected node.

In some embodiments, the data elements of the dataset 102 are connected using a nearest neighbor graph algorithm. Simply put, distances between each pair of data elements of the dataset 102 are computed to determine if they are to be connected. The distance is computed using the d-dimensional feature attributes of each data element. In a d-dimensional Euclidean space, all points at distance epsilon from a given sample x lie in the hyper-cube. For example, in 3-dimensions, a cube centered at a specific point of choice (i.e., a selected data element). All possible neighbors of that data element at distance epsilon are considered to be neighbors of the selected data element, and so the hypercube centered at the selected data element has length epsilon.

The data is fitted with such a model (holding out the test set for subsequent evaluations) and the learned parameters of the following distributions are stored: the prior class distribution p(C=c); the mixture weights distribution p(M=m|C=c); and the emission distribution p(X=x|M=m,C=c).

The prior class and mixture weights distributions are categorical, and therefore parametrized by a conditional probability table (CPT), i.e., a matrix of values whose size depends on the cardinality of M and C. The emission distribution is Gaussian, and is parametrized by a mean vector of d dimensions and a covariance matrix of dimension d×d. If a diagonal covariance matrix is chosen, the parametrization reduces to only d parameters, which are on the diagonal.

HNB is fitted using a maximum likelihood criterion to learn the parameters of the above-identified distributions from the dataset 102. There are at least two possible ways for that: a classical one relies on Expectation-Maximization algorithm, and a second one relies on Variational Inference, but one might also use backpropagation. But, generally speaking, the process to fit/learn an HNB is to maximize the likelihood of the data.

For example, P(C=c) is the probability that any sample of the dataset 102 has class c. P(M=m|C=c) is the conditional probability that a sample of class c is generated from the component m of the Gaussian mixture. The emission distribution is the probability that the features of the data element are x given that the sample has been generated from the component m and has class c.

Based on the prior class, mixture weight, and emission distributions, the “best” nearest neighbor graph can be estimated in terms of distance between neighbors. After this estimate is obtained, the nearest neighbor algorithm can be applied to obtain a graph that satisfies this distance between samples of the original dataset.

Compared to the computationally expensive job of trying many different graph constructions and then computing the CCNS matrix, the HNB model and its distributions are exploited to estimate how the CCNS matrix will look like when applying a generic distance-based nearest neighbor algorithm construction, without having to explicitly construct a graph each time. The best nearest neighbor graph is determined by finding the best length of the hypercube. This is done by iteratively trying many different length and efficiently estimating the resulting CCNS.

A value in the CCNS matrix indicates the Euclidean distance between each pair of classes (identified by a row/column pair) in terms of neighborhood class dissimilarity. A good CCNS matrix means that the values of the Euclidean distance lying on the diagonal are low whereas the other values are high.

After the data is fitted with the model, and neighbors for each data node are determined, the computing device 104 determines learned parameters of the following distributions: the prior class distribution p(C=c); the mixture weights distribution p(M=m|C=c); and the emission distribution p(X=x|M=m,C=c).

In step 206, the method 200 includes finding (e.g., determining) the best theoretical cross-class neighborhood similarity distance (e.g., how similar the neighborhoods of two distinct nodes are in terms of class label distribution). The learned parameters are used to compute a theoretical approximation of the cross-class neighborhood similarity under the nearest neighbor graph to look for “good” cross-class neighborhood similarity. In some embodiments, the cross-class neighborhood similarity uses the Euclidian distance as a similarity metric, so a promising structure would have low intra-class cross-class neighborhood similarity distance and high inter-class cross-class neighborhood similarity distance. For example, the cross-class neighborhood similarity can be formalized as laid out below:

Given a graph g, the cross-class neighborhood similarity between classes c, c′∈ [symbol] is given by

$${s ( c , {c}^{′} )}^{d} {=}^{def} {𝔼}_{p ( x \vert c ) p ( {x}^{′} \vert {c}^{′} )} [ Ω ( {q}_{c} ( x ) , {q}_{{c}^{′}} ( {x}^{′} ) ) ] \qquad ( 1 )$$

where Ω computes a similarity score between vectors and the function q_{c}: [symbol] ^{D}→ [symbol] (resp q_{c′}) computes, for every c″∈ [symbol] , the probability vector that a node of class c (resp. c′) with attributes x has a neighbor of class c″.

In some embodiments, the Euclidean distance as can be used as function. The lower bound of Equation 1 follows from Jensen's inequality, since every norm is convex, and from the linearity of expectation:

$$( 2 )$$

$${s ( c , {c}^{′} ) \geq {‖ {𝔼}_{p ( x \vert c ) p ( {x}^{′} \vert {c}^{′} )} [ ( {q}_{c} ( x ) - {q}_{{c}^{′}} ( {x}^{′} ) ] ‖}_{2} = {𝔼}_{p ( x \vert c )} [ ( {q}_{c} ( x ) ] {𝔼}_{p ( {x}^{′} \vert {c}^{′} )} [ {q}_{{c}^{′}} ( {x}^{′} ) ] ‖}_{2} .$$

This bound assigns non-zero values to the inter-class neighborhood similarity, whereas Monte Carlo approximations of Equation 1 estimate the intra-class similarity.

The theoretical approximation is parametrized by ε, which indicates how far, on average, neighbors are assumed to be. The theoretical cross-class neighborhood similarity, is approximated via Monte Carlo simulations using the fitted distributions of step 2 and the following theoretical approximation of the q_{c}, which are computed as follows:

Given a hypercube length ε and class c′∈ [symbol] , the unnormalized probability that a sample of class c′ has a neighbor of class c is defined as:

$${{M}_{{c}^{′}} ( c )}^{d} {=}^{def} {𝔼}_{x p ( x \vert {c}^{′} )} [ {M}_{x} ( c ) ] \qquad ( 5 )$$

It is still possible to compute M_{c′}(c) in closed form, which is shown below.

$$( 6 )$$

$${M}_{{c}^{′}} ( c ) = p ( c ) \text{} {\sum }_{m , {m}^{′}}^{\vert ℳ \vert} p ( {m}^{′} \vert {c}^{′} ) p ( m \vert c ) {\prod }_{f = 1}^{D} ( Φ ( \frac{{μ}_{{c}^{′} {m}^{′} f} + \frac{ε}{2} - {μ}_{cmf}}{\sqrt{{σ}_{cmf}^{2} + {σ}_{{c}^{′} {m}^{′} f}^{2}}} ) - Φ ( \frac{{μ}_{{c}^{′} {m}^{′} f} - \frac{ε}{2} - {μ}_{cmf}}{\sqrt{{σ}_{cmf}^{2} + {σ}_{{c}^{′} {m}^{′} f}^{2}}} ) ) .$$

Given a class c′ and c∈R, the expected class distribution around samples of class c′ is modeled by the categorical random variable D_{c′}, such that:

$$p ( {D}_{{c}^{′}} = c ) = {p}_{{c}^{′}} ( c ) \qquad ( 7 )$$

$$with$$

$${p}_{{c}^{′}} ( c ) = \frac{{M}_{{c}^{′}} ( c , ε )}{{\sum }_{i = 1}^{\vert C \vert} {M}_{{c}^{′}} ( i , ε )} .$$

where p_{c} is used as a replacement for q_{c} to approximate the cross-class neighborhood similarity via Monte Carlo simulations.

In step 208, the method includes building a nearest neighbor graph based on the best theoretical cross-class neighborhood similarity distance. For example, the computing device 104 can efficiently compute the CCNS approximation for different values of E, and pick the one that returns the best trade-off between intra- and inter-class distances. At this point, a nearest neighbor graph is built where the neighbors of each sample lie in the hypercube of length ε centered at the sample's attributes. In some embodiments, the attributes of the data elements can be continuous and discrete features. In some cases, discrete features are interpreted as continuous numbers. In this way, the connectivity is built just once, rather than explicitly trying many different alternatives, which would have a high computational burden and memory requirements.

In some embodiments, the different values of E are spread over a range. The range starts from close to zero, and is gradually increased until the CCNS becomes uninformative or until the quality of the CCNS stops increasing. Because the maximum distance of a neighbor is optimized instead of the number of nodes, each value of E can yield a different number of neighbors for each node.

For example, using the learned parameters and the determined best theoretical cross-class neighborhood similarity distance, the computing device 104 instructs the graph builder 106 to construct a nearest neighbor graph. In some embodiments, the nearest neighbor graph is built by placing each data point in the tabular dataset 102 at a center of a hypercube. The hypercube has an edge E. The neighbors of the selected data point are within the hypercube that is constructed around the selected node at the center. The edge of the hypercube is optimized based on the tradeoff between the intra-class and inter-class distances of the selected node, and the nearest neighbor graph is constructed based on the optimized edge of the hypercube.

In step 210, the method includes training a graph machine learning method. With an artificial structure in place, a graph machine learning classifier can be trained to predict the class of each entity based on a structure that should, in principle, be sensible for the class separability of the different samples. In fact, samples of the same class can enjoy a similar neighborhood label distribution (it usually implies a similar neighborhood attributes' distribution), whereas samples of distinct classes can likely have a different neighborhood distribution and therefore can be easier to separate. However, it is advantageously possible to use the same method to tackle any subsequent machine learning task regardless of the previous availability of class information. Thus, embodiments of the present invention can be practically applied to improve the accuracy and reduce the computational burden of a number of machine learning tasks. For example, embodiments of the present invention can be applied to automated healthcare, AI drug development, material design and predictive maintenance.

For instance, once the graph builder 106 generates the nearest neighbor graph, the computing device 104 instructs training component 108 to train a graph machine learning classifier to predict a class of different elements of data that are added to the dataset 102. Once classified, data points of dataset 102 that are part of the same class are assigned similar label attributes, whereas samples of different classes can have different label attributes.

In one embodiment, the present invention can be applied to the machine learning task of predicting clinical risk of patients. Predicting clinical risk in hospitals using AI can accelerate diagnoses of patients that could be subject to determined illnesses (for example, sepsis, acute kidney injuries, etc.). However, hospitals usually rely on relatively simple electronic health records (EHRs) which are represented as tabular data. An embodiment of the present invention can be used to first build a graph of the patients and then used to make more accurate predictions than structure-agnostic methods. Here, the data source includes patient data, which consists of a table of attributes for each patient, for example the EHRs. This data includes, but it is not limited to, basic vital measurements and laboratory exams such as heart rate, oxygen saturation, weight, height, glucose, temperature, pH etc. Application of the method according to an embodiment of the invention can construct a sensible graph structure based on the theoretical approximation of the CCNS, and then apply a graph machine learning method to predict the clinical risk of each patient. The output can be a graph of patient connectivity and prediction of clinical risk.

For example, computing device 104 of the system can access the patient health records stored in tabular form in dataset 102 and construct a nearest neighbor graph using the data. In order to construct the nearest neighbor graph, the computing device 104 can instruct graph builder 106 to access the data from the dataset 102. The data in the dataset 102 can include patient data related to vital measurements, laboratory test results, and crucial patient identifying data. A model can be applied to the data of the dataset 102 and graph builder 106 can extract learned parameters from the application of the model to the dataset 102. The computing device 104 can also instruct graph builder 106 to determine the best theoretical cross-class neighborhood similarity distance from the data of the dataset 102 by optimizing the intra-class and inter-class distances. Based on the best theoretical cross-class neighborhood similarity distance and the learned parameters, a neighborhood graph is constructed, that is used to train a graph machine learning classifier using training component 108 in order to perform predictions of clinical risk for patients, the data for which is input into the dataset 102.

In another embodiment, the present invention can be applied to the machine learning task of patient stratification: predicting the response to treatment in clinical trials. One of the factors which mostly impacts clinical trials' budgets, and ultimately their outcome, is the patient selection process. Many times patients who should not be eligible for the treatment (for example, because the trial will not cause a positive response) are enrolled anyway, and this can and ultimately lead to the reduce effectiveness failure of the trial itself. The rationale is to perform a stratification of patients using information at a genetic level, to predict earlier their response, which can result in a stratification between responders and non-responders. Given tabular data of patients, it is possible to predict the response for the other patients while also identifying an “ideal candidate” for it. Here, the data source includes patients with genomic activity information (for example, single cell RNA-sequencing), their response to a specific drug, and publicly available ontologies. Application of the method according to an embodiment of the present invention can construct a sensible graph structure based on the theoretical approximation of the CCNS, and then apply a graph machine learning method to predict the clinical risk of each patient. The output can be a graph of patient connectivity and prediction of the patient's response to the drug (low/medium/high).

Similarly, the computing device 104 of the system can use the above disclosed method for patient stratification. The patient stratification can be used to predict patient response to treatment in clinical trials. In such cases, the patient information that can be stored in the dataset 102 can include genetic information related to patients. The genetic information related to the patients can be accompanied with their response to specific drugs. A model can be applied to this information stored in the dataset 102 to determine a theoretical cross-class neighborhood similarity and learned parameters, which can be used to generate a nearest neighbor graph. The nearest neighbor graph can be used to train a graph training model to determine which incoming patients can be best suited for an upcoming drug trial.

In another embodiment, the present invention can be applied to predictive maintenance, for example for the machine learning task of determining low/high quality soil areas using sensors. Precision agriculture seeks to improve productivity while reducing costs using smart devices that constantly monitor the target environment. Soil monitoring is one example of how sensors can be used to determine where and when it is necessary to provide maintenance, for instance, irrigate the soil, in order to keep humidity and temperature to optimal levels. High-quality predictions are important for efficient predictive maintenance, and considering the surrounding areas of the soil allows to make more informed predictions. By finding the “right” distance to maximize the classification performances, an embodiment of the present invention can build a nearest neighbor graph of soil areas such that a subsequent graph machine learning predictor can classify each area. Here, the data source includes a sensor for each soil area, which consists of data regarding humidity, temperature, and other metrics of interest related to the soil. Application of the method according to an embodiment of the present invention first determines an approximation for the best nearest neighbor graph distance between soil areas for the task. Then, a subsequent graph machine learning classifier is trained and evaluated on the graph, predicting the quality (for example, low/medium/high) of all soil areas considered. The output can be a connectivity structure between soil areas and classification of their soil quality (for example, low/medium/high).

In some embodiments, the system 100 can be used to perform predictive maintenance in the field of precision agriculture. In such embodiments, dataset 102 can include information related to soil, such as areas, properties and other metrics that are related to the soil. A data model is applied to the soil data stored in dataset 102, after which learned parameters and a theoretical cross-class neighbor similarity distance are determined. Upon optimization of the theoretical cross-class neighbor similarity distance, a nearest neighbor graph is constructed that is used to train a graph machine learning model that is able to output a connectivity structure between soil areas and classification of their soil quality (for example, low/medium/high) based on information in the dataset 102.

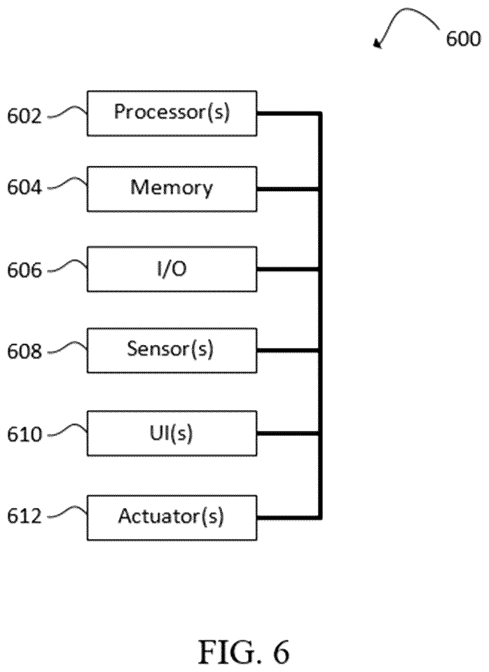

Referring to FIG. 6, a processing system 600 can include one or more processors 602, memory 604, one or more input/output devices 606, one or more sensors 608, one or more user interfaces 610, and one or more actuators 612. Processing system 600 can be representative of each computing system disclosed herein.

Processors 602 can include one or more distinct processors, each having one or more cores. Each of the distinct processors can have the same or different structure. Processors 602 can include one or more central processing units (CPUs), one or more graphics processing units (GPUs), circuitry (e.g., application specific integrated circuits (ASICs)), digital signal processors (DSPs), and the like. Processors 602 can be mounted to a common substrate or to multiple different substrates.

Processors 602 are configured to perform a certain function, method, or operation (e.g., are configured to provide for performance of a function, method, or operation) at least when one of the one or more of the distinct processors is capable of performing operations embodying the function, method, or operation. Processors 602 can perform operations embodying the function, method, or operation by, for example, executing code (e.g., interpreting scripts) stored on memory 604 and/or trafficking data through one or more ASICs. Processors 602, and thus processing system 600, can be configured to perform, automatically, any and all functions, methods, and operations disclosed herein. Therefore, processing system 600 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 600 can be configured to perform task “X”. Processing system 600 is configured to perform a function, method, or operation at least when processors 602 are configured to do the same.

Memory 604 can include volatile memory, non-volatile memory, and any other medium capable of storing data. Each of the volatile memory, non-volatile memory, and any other type of memory can include multiple different memory devices, located at multiple distinct locations and each having a different structure. Memory 604 can include remotely hosted (e.g., cloud) storage.

Examples of memory 604 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 604.

Input-output devices 606 can include any component for trafficking data such as ports, antennas (i.e., transceivers), printed conductive paths, and the like. Input-output devices 606 can enable wired communication via USB®, DisplayPort®, HDMI®, Ethernet, and the like. Input-output devices 606 can enable electronic, optical, magnetic, and holographic, communication with suitable memory 606. Input-output devices 606 can enable wireless communication via WiFi®, Bluetooth®, cellular (e.g., LTE®, CDMA®, GSM®, WiMax®, NFC®), GPS, and the like. Input-output devices 606 can include wired and/or wireless communication pathways.

Sensors 608 can capture physical measurements of environment and report the same to processors 602. User interface 610 can include displays, physical buttons, speakers, microphones, keyboards, and the like. Actuators 612 can enable processors 602 to control mechanical forces.

Processing system 600 can be distributed. For example, some components of processing system 600 can reside in a remote hosted network service (e.g., a cloud computing environment) while other components of processing system 600 can reside in a local computing system. Processing system 600 can have a modular design where certain modules include a plurality of the features/functions shown in FIG. 6. For example, I/O modules can include volatile memory and one or more processors. As another example, individual processor modules can include read-only-memory and/or local caches.

In an embodiment, the present invention provides a method for building a nearest neighbor graph using a theoretical approximation of the cross-class neighborhood similarity, the method comprising the steps of:
1) Tabular data collection (see FIG. 2, step 202).
2) Fitting a Hierarchical Naïve Bayes model to the data, in order to approximate the distributions of the true data generation process (see FIG. 2, step 204).
3) Using the distributions to compute different approximation of the CCNS with respect to the parameter F and pick the one that induces the best trade-off between intra- and inter-class CCNS (see FIG. 2, step 206).
4) Building a nearest neighbor graphs such that the neighbors of each node lie in the hypercube of length F centered at the sample (see FIG. 2, step 208).
5) Training and evaluating the graph machine learning method on top of the graph to solve predictive tasks about the samples (see FIG. 2, step 210).

Embodiments of the present invention provide for the following improvements over existing technology:
1. Providing a method that efficiently approximates the cross-class neighborhood similarity matrix by first fitting a Hierarchical Naïve Bayes model and then using the learned parameters to infer the best nearest neighbor graph structure. (see steps 2) and 3) of the preceding method).
2. By developing a distribution that computes the probability that a node of a given class has a nearest neighbor of another class, providing to approximate the CCNS without computing a graph, and therefore efficiently transforming a tabular classification problem into a node classification problem to be solved with graph machine learning techniques (see step 3) of the preceding method).

According to existing technology, there is no current method that allows to estimate the CCNS without first creating a graph. In the case of nearest neighbor graphs, embodiments of the present invention enable to avoid the creation of such graphs, which saves computational time and resources, and approximately choose a good connectivity structure based on theoretical results. In the worst case, a nearest neighbor search can be quadratic in the number of entities, whereas this is not an issue according to embodiments of the present invention.

In the following, further background and description of exemplary embodiments of the present invention, which can overlap with some of the information provided above, are provided in further detail. To the extent the terminology used to describe the following embodiments can differ from the terminology used to describe the preceding embodiments, a person having skill in the art would understand that certain terms correspond to one another in the different embodiments. Features described below can be combined with features described above in various embodiments.

Researchers have used nearest neighbor graphs to transform classical machine learning solve problems on tabular data into node classification tasks to with graph representation learning methods. Such artificial structures often reflect the homophily assumption, believed to be a key factor in the performances of deep graph networks. In light of recent results demystifying these beliefs, a theoretical framework is introduced to understand the benefits of nearest neighbor graphs when a graph structure is missing. Cross-Class Neighborhood Similarity (CCNS) is formally analyzed, used to evaluate the usefulness of structures, in the context of nearest neighbor graphs. Moreover, the class separability induced by deep graph networks on a k-NN graph is formally studied. Quantitative experiments demonstrate that, under full supervision, employing a k-NN graph might not offer benefits compared to a structure-agnostic baseline. Qualitative analyses suggest that the framework is good at estimating the CCNS and hint at k-NN graphs never being useful for such tasks, thus advocating for the study of alternative graph construction techniques.

The pursuit of understanding real-world phenomena has often led researchers to model the system of interest as a set of interdependent constituents, which influence each other in complex ways. In disciplines such as chemistry, physics, and network science, graphs are a convenient and well-studied mathematical object to represent such interacting entities and their attributes. In machine learning, the term “graph representation learning” refers to methods that can automatically leverage graph-structured data to solve tasks such as entity (or node), link, and whole-graph predictions.

Most of these methods assume that the relational information, that is the connections between entities, naturally emerges from the domain of the problem and is thus known. There is also broad consensus that connected entities typically share characteristics, behavioral patterns, or affiliation, something known as the homophily assumption. This is possibly why, when the structure is not available, researchers have tried to artificially build Nearest Neighbor graphs from tabular data, by connecting entities based on some attribute similarity criterion, with applications in healthcare, fake news and spam detection, biology, and document classification. From an information-theoretic perspective, the creation of such graphs does not add new information as it depends on the available data; that said, what makes their use plausible is that the graph construction is a form of feature engineering that often encodes the homophily assumption. Combined with the inductive bias of Deep Graph Networks (DGNs), this strategy aims at improving the generalization performances on tabular data compared to structure-agnostic baselines, for example, a Multi-Layer Perceptron (MLP).

Indeed, using a k-nearest neighbor graph has recently improved the node classification performances under the scarcity of training labels. This is also known as the semi-supervised setting, where one can access the features of all nodes but the class labels are available for a handful of those. A potential explanation for these results is that, by incorporating neighboring values into each entity's representation, the neighborhood aggregation performed by deep graph networks acts as a regularization strategy that prevents the classifier from overfitting the few labeled nodes. However, it is still unclear what happens when one has access to all training labels (hereinafter the fully-supervised setting), namely if these graph-building strategies grant a statistically significant advantage in generalization compared to a structure-agnostic baseline. In this respect, proper comparisons against such baselines are often lacking or unclear in previous works, an issue that has also been reported in recent papers about the reproducibility of node and graph classification experiments.

In addition, it was recently shown that homophily is not required to achieve good classification performances in node classification tasks; rather, what truly matters is how much the neighborhood class label distributions of nodes of different classes differ. This resulted in the definition of the empirical Cross-Class Neighborhood Similarity (CCNS), an object that estimates such similarities based on the available connectivity structure. Yet, whether or not artificially built graphs can be useful for the task at hand has mainly remained an empirical question, and more theoretical conditions for which this happens are still not understood.

As described herein, a framework is introduced to approach this question, and two analyses of independent interest are provided. Inspired by the cross-class neighborhood similarity, a first embodiment studies the neighboring class label distribution of nearest neighbor graphs. A second embodiment deals with the distribution of entity embeddings induced by deep graph neural networks on a k-nearest neighbor graph, and it is used to quantify class separability in both the input and the embedding spaces. Overall, the results suggest that building a k-nearest neighbor graph might not be a good idea. To validate with empirical evidence, four baselines across 11 tabular datasets are compared to check that the k-nearest neighbor graph construction does not give statistically significant advantages in the fully-supervised setting. In addition, the learning of data distributions that would make a k-nearest neighbor graph useful in practice is reverse engineered. From the empirical results, it is understood that this is never the case. Therefore, there is a need for alternative graph construction techniques.

In summary: i) under some assumptions on the data generative process, the cross class neighborhood for nearest neighbor graphs is estimated and a first lower bound is provided; ii) the effects of applying a simple deep graph network to an artificial k-nearest neighbor graph on the class separability of the input data are studied; iii) a robust comparison between structure-agnostic and structure-aware baselines on a set of 11 datasets that validate the theoretical results is performed; iv) qualitative analyses further suggest that using the k-nearest neighbor graph might not be advisable.

The early days of graph representation learning date back to the end of the previous century, when backpropagation through structures was developed for directed acyclic graphs. These ideas laid the foundations for the adaptive processing of cyclic graphs by the recurrent graph neural network and the feedforward neural network for graphs, which laid the foundations of today's deep graph networks. Both methods iteratively compute embeddings of the graphs' entities (also called nodes) via a local message passing mechanism that propagates the information through the graph. In recent years, many neural and probabilistic deep graph networks have emerged bridging ideas from different fields of machine learning. In some embodiments, the analysis is setup in the context of these message-passing architectures. Even more recently, transformer models have begun to appear in graph-related tasks as well. Akin to kernel methods for graphs, this class of methods mainly relies on feature engineering to extract rich information from the input graph, and some perform very well at molecular tasks. However, the architecture of (graph) transformers is not intrinsically more powerful than deep graph networks, and their effectiveness depends on the specific encodings used. Therefore, gaining a better understanding of the inductive bias of deep graph networks remains a compelling research question.

The construction of nearest neighbor graphs found recent application in predicting the mortality of patients, by connecting them according to specific attributes of the electronic health records. In addition, it was used in natural language processing to connect messages and news with similar contents to tackle spam and fake news detection, respectively. In both cases, the authors computed similarity based on some embedding representation of the text, whereas the terms' frequency in a document was used previously as a graph-building criterion for a generic document classification task. Finally, k-nearest neighbor graphs have also been built based on chest computerized tomography similarity for early diagnoses of COVID-19.

Most of the works on deep graph networks deal with the problems of over-smoothing and over-squashing of learned representations, as well as the discriminative power of such models. In this context, it was also believed that deep graph networks based on message passing perform favorably for homophilic graphs and not so much for heterophilic ones. However, recent works suggest a different perspective; the generalization performances depend more on the neighborhood distributions of nodes belonging to different classes and on a good choice of the model's weights. The cross-class neighborhood similarity was recently proposed as an effective (but purely empirical) strategy to understand if a graph structure is useful or not for a node classification task. Inspiration is taken from the class neighborhood similarity to study the behavior of the neighborhood class label distributions around nodes and compute the first lower bound of the class neighborhood similarity for nearest neighbor graphs.

Structure learning and graph rewiring are also related but orthogonal topics. Rather than pre-computing a fixed structure, these approaches discover dependencies between samples and can enrich the original graph structure when this is available. They have been applied in contexts of scarce supervision, where a k-nearest neighbor graph proved to be a powerful baseline when combined with deep graph networks. At the same time, the combinatorial nature of graphs makes it difficult and expensive to explore the space of all possible structures, making the a priori construction of the graph a sensible alternative.

In accordance with some embodiments, background notions and assumptions that can be useful throughout the analysis are introduced. The starting point is a classification task over a set of classes [symbol] , where each sample u is associated with a vector of attributes xᵤ∈ [symbol] ^{D}, D∈ [symbol] and a target class label yᵤ∈ [symbol] .

A graph g of size N is a quadruple ( [symbol] , ξ, X, [symbol] ), where [symbol] ={1, . . . , N} represents the set of nodes and E is the set of directed edges (u, v) from node u to node v. The symbol X={Xᵤ, ∀u∈ [symbol] } defines the set of i.i.d. random variables with realizations {xᵤ∈ [symbol] ^{D}, ∀u∈ [symbol] }. The same definition applies to the set of target variables [symbol] ={Yᵤ, ∀u∈ [symbol] } and their realizations {yᵤ∈C, ∀u∈ [symbol] }. The symbol [symbol] _{c} denotes the subset of nodes with target label c∈ [symbol] ; and the neighborhood of node u is defined as [symbol] ={v∈ [symbol] |(v,u)∈ξ}.

A Gaussian (or normal) univariate distribution with mean and variance μ, σ²∈ [symbol] is represented as [symbol] (·;μ, σ²), using μ∈ [symbol] ^{D}∈ [symbol] ^{D×D} for the multivariate case. The probability density function (p.d.f.) of a univariate normal random variables parametrized by μ, σ is denoted by ϕ(·) together with its cumulative density function (c.d.f)

$$F ( w ) = Φ ( \frac{w - μ}{σ} ) = \frac{1}{2} ( 1 + erf ( \frac{w - μ}{σ √ 2} ) ) ,$$

where erf is the error function. Subscripts can denote quantities related to a specific random variable.

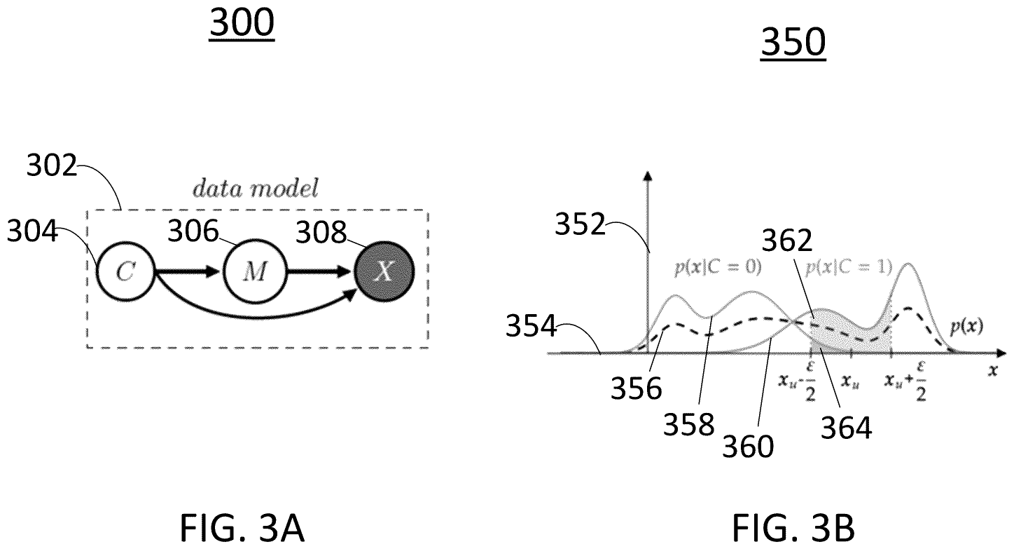

The method transforms the initial task into a node classification problem, where the different samples become the nodes of a single graph, and the edges are computed by some nearest-neighbor algorithm. It is assumed the true data distribution p(X=x) is defined by the hierarchical graphical model of FIG. 3A, with the | [symbol] | latent classes modeled by a categorical random variables C with prior distribution p(C=c) and |M| mixtures for each class modeled by M˜p(M=m|C=c). It is further provided that the attributes (i.e., the realization of a random variables) are conditionally independent when the class c and the mixture m are known, i.e., p(X=x)= [symbol] p(c)Σₘ₌₁^{|M|}p(m|c)Π_{f=1}^{D}p(x_{f}|m,c). This graphical model allows consideration of continuous attributes and, to some extent, categorical values. FIG. 3B depicts an example of one such data distribution for D=1. Hereinafter, p(X_{f}=x_{f}|m,c)= [symbol] (x_{f};μ_{cmf},σ²_{cmf}) is obtained, and for notational convenience one can write p(x|m,c)= [symbol] (x;μ_{mc},Λ_{mc}) with diagonal covariance Λ_{mc}=diag (σ_{mc}_{₁}², . . . , σ_{mc}_{_{D}}²). Finally, it is assumed that the neighboring class labels of a node are independent from each other. In some embodiments, FIG. 3B a visual example of a data distribution generated by the graphical model, with an intuitive visualization of the class posterior mass around a point xᵤ.

FIG. 3A represents the assumptions on the data distribution through a graphical model 302. For example, i.e., a hierarchical mixture of distributions of data model 302 includes observed variables 308 and latent variables 304 and 306.

To refer to the surroundings of a point in space, the notion of hypercubes is used. A hypercube of dimension D centered at point x∈ [symbol] ^{D} of side length ε is the set of points given by the Cartesian product

$${H}_{ε} ( x ) = [ {x}_{1} - \frac{ε}{2} , {x}_{1} + \frac{ε}{2} ] \times … \times [ {x}_{D} - \frac{ε}{2} , {x}_{D} + \frac{ε}{2} ] .$$

The cross-class neighborhood similarity computes how similar the neighborhoods of two distinct nodes are in terms of class label distribution, and it provides an aggregated result over pairs of target classes. Intuitively, if nodes belonging to distinct classes happen to have similar neighboring class label distributions, then it can be unlikely that a classifier can correctly discriminate between these two nodes after a message passing operation because the nodes' embeddings can look very similar. On the other hand, nodes of different classes with very different neighboring class label distributions can be easier to separate. This intuition relies on the assumption that nodes of different classes typically have different attributes.

The cross-class neighborhood similarity is formalized as:

Definition 3.1 (Cross Class Neighborhood Similarity). Given a graph g, the cross-class neighborhood similarity between classes c, c′∈ [symbol] is given by

$$s ( c , {c}^{′} ) {=}^{def} {𝔼}_{p ( x \vert c ) p ( {x}^{′} \vert {c}^{′} )} [ Ω ( {q}_{c} ( x ) , {q}_{{c}^{′}} ( {x}^{′} ) ) ] \qquad ( 1 )$$

where Ω computes a similarity score between vectors and the function q_{c}: [symbol] ^{D}→ [symbol] (resp q_{c′}) computes the probability vector that a node of class c (resp. c′) with attributes x (resp. x′) has a neighbor of class c″, for every c″∈ [symbol] .

The definition of q_{c} and q_{c′} is the key ingredient of Equation 1. In the following, it is shown that it is possible to analytically compute these quantities when it is assumed to be a nearest-neighbor structure. With a loose definition of “nearest” all existing nodes can be included, but it is also shown that doing that corresponds to a crude approximation of the quantities of interest.

From now on, the Euclidean distance as can be used as function. The lower bound of Equation 1 follows from Jensen's inequality, since every norm is convex, and from the linearity of expectation:

$$s ( c , {c}^{′} ) \geq {‖ {𝔼}_{p ( x \vert c ) p ( {x}^{′} \vert {c}^{′} )} [ {q}_{c} ( x ) - {q}_{{c}^{′}} ( {x}^{′} ) ] ‖}_{2} = {‖ {𝔼}_{p ( x \vert c )} [ {q}_{c} ( x ) ] {𝔼}_{p ( {x}^{′} \vert {c}^{′} )} [ {q}_{{c}^{′}} ( {x}^{′} ) ] ‖}_{2} . \qquad ( 2 )$$

This bound assigns non-zero values to the inter-class neighborhood similarity, whereas Monte Carlo approximations of Equation 1 estimate the intra-class similarity.

The class label distribution in the surroundings of some node u are studied first. In the example of FIG. 3B, a binary classification problem with | [symbol] |=2 is considered and depicts the conditional distributions p(xᵤ|C=0),p(xᵤ|C=1) with curve 358 and curve 360, respectively. Dashed black line 356, instead, represents p(x) assuming a non-informative class prior. If the neighbors of u belong to the hypercube H_{ε}(xᵤ) for some ε, then the probability that a neighbor can belong to class c depends on how much class-specific probability mass, i.e., the shaded areas 364 and 362, there is in the hypercube. Since the shaded area 362 is larger than the shaded area 364, finding a neighbor of class 1 is more likely to happen. Formally, the probability of a neighbor belonging to class c in a given hypercube is defined as the weighted posterior mass of C contained in that hypercube.

Definition 3.2 (Posterior Mass Mₓ(c) Around Point x). Given a hypercube H_{ε}(x) centered at point x∈ [symbol] ^{D}, and a class c∈ [symbol] , the posterior mass Mₓ(c) is the unnormalized probability that a point in the hypercube has class c:

$$\begin{matrix}{M}_{x} ( c , ε ) {=}^{def} {𝔼}_{w ∈ {H}_{ε} ( x )} [ p ( c \vert w ) ] = {\int }_{w ∈ {H}_{ε} ( x )} p ( c \vert w ) p ( w ) dw \\ = p ( c ) {\int }_{w ∈ {H}_{ε} ( x )} p ( w \vert c ) dw\end{matrix} , \qquad ( 3 )$$

where the last equality follows from Bayes' theorem.

When clear from the context, the argument F can be omitted from all quantities of interest to simplify the notation. The following proposition shows how to compute Mₓ(c) analytically. Proofs are included below.

Equation 3 has the following analytical form

$${M}_{x} ( c ) = p ( c ) {\sum }_{m}^{\vert ℳ \vert} p ( m \vert c ) {\prod }_{f = 1}^{D} ( {F}_{{Z}_{cmf}} ( {x}_{f} + \frac{ε}{2} ) - {F}_{{Z}_{cmf}} ( {x}_{f} - \frac{ε}{2} ) ) \qquad ( 4 )$$

where Z_{cmf} is a random variables with Gaussian distribution p(w|m,c)= [symbol] (w;μ_{cmf},σ_{cmf}²).

To reason about an entire class rather than individual samples, therefore being able to compute the two quantities on the right-hand side of Equation 2, the previous definition is extended by taking into account all samples of class c′∈ [symbol] . Thus, the method computes the average probability that a sample belongs to class c in the hypercubes centered around samples of class c′.

(Expected Class c Posterior Mass M_{c′}(c) for Samples of Class c′). Given a hypercube length ε and a class c′∈ [symbol] , the unnormalized probability that a sample of class c′ has a neighbor of class c is defined as

$${M}_{{c}^{′}} ( c ) {=}^{def} {𝔼}_{x \sim p ( x \vert {c}^{′} )} [ {M}_{x} ( c ) ] . \qquad ( 5 )$$

It is still possible to compute M_{c′}(c) in closed form, which is shown below.

Equation 5 has the following analytical form

$${M}_{{c}^{′}} ( c ) = \qquad ( 6 )$$

$$p ( c ) {\sum }_{m , {m}^{′}}^{\vert ℳ \vert} p ( {m}^{′} \vert {c}^{′} ) p ( m \vert c ) {\prod }_{f = 1}^{D} ( Φ ( \frac{{μ}_{{c}^{′} {m}^{′} f + \frac{ε}{2} - {μ}_{cmf}}}{\sqrt{{σ}_{cmf}^{2} + {σ}_{{c}^{′} {m}^{′} f}^{2}}} ) - Φ ( \frac{{μ}_{{c}^{′} {m}^{′} f - \frac{ε}{2} - {μ}_{cmf}}}{\sqrt{{σ}_{cmf}^{2} + {σ}_{{c}^{′} {m}^{′} f}^{2}}} ) ) .$$

Based on the above, it is possible to determine how much class-c posterior probability mass is available, on average, around samples of class c′. To get a proper class c′-specific distribution over neighboring class labels, a normalization step is applied using the fact that M_{c′}(c)≥0 ∀c∈ [symbol] .

In some embodiments, a ε-Neighboring Class Distribution is disclosed. Given a class c′ and an ε∈ [symbol] , the neighboring class distribution around samples of class c′ is modeled as

$${p}_{{c}^{′}} ( c ) = \frac{{M}_{{c}^{′}} ( c , ε )}{{\sum }_{i = 1}^{\vert 𝒞 \vert} {M}_{{c}^{′}} ( i , ε )} \qquad ( 7 )$$

This distribution formalizes the notion that, in a neighborhood around points of class c′, the probability that points belong to class c does not necessarily match the true prior distribution p(C=c). However, this becomes false when infinitely-large hyper-cube is considered.

In some embodiments, the first D derivatives of [symbol] M_{c′}(i) can be different from 0 in an open interval I around ε=0. Then Equation 7 has the following limits:

$$\begin{matrix}{lim}_{ε \to 0} {p}_{{c}^{′}} ( c ) = \frac{p ( c ) {\sum }_{m , {m}^{′}}^{\vert ℳ \vert} p ( m \vert c ) p ( {m}^{′} \vert {c}^{″} ) {\prod }_{f = 1}^{D} {ϕ}_{{Z}_{{cmm}^{′} f}} ( 0 )}{{\sum }_{i}^{\vert 𝒞 \vert} p ( i ) {\sum }_{m , {m}^{′}}^{\vert ℳ \vert} p ( m \vert c ) p ( {m}^{′} \vert {c}^{″} ) {\prod }_{f = 1}^{D} {ϕ}_{{Z}_{{imm}^{′} f}} ( 0 )} , \\ {lim}_{ε \to ∞} {p}_{{c}^{′}} ( c ) \\ = p ( c )\end{matrix} \qquad ( 8 )$$

where Z_{imm′f} has distribution [symbol] (·;−a_{imm′f},b_{imm′f}²),

$${a}_{{imm}^{′} f} = 2 ( {μ}_{{c}^{′} {m}^{′} f} - {μ}_{imf} ) and {b}_{{imm}^{′} f} = 2 \sqrt{{σ}_{imf}^{2} + {σ}_{{c}^{′} {m}^{′} f}^{2}} .$$

The choice of ε, which intuitively encodes definition of “nearest” neighbor, plays a crucial role in determining the distribution of a neighbor's class label. When the hypercube is too big, the probability that a neighbor has class c matches the true prior p(c) regardless of the class c′, i.e., a crude assumption is made about the neighbor's class distribution of any sample. If instead, a smaller hypercube is considered, a less trivial behavior is observed and the probability p_{c′}(c) is directly proportional to the distance between the means p_{c′m′} and μ_{cm}, as one would intuitively expect for simple examples.

To summarize, p_{c}′(c) can be used as an approximation for [symbol] _{p(x|c′)}[q_{c}′(x)] of Equation 2; similarly, a normalized version of M_{c}(x) can be used in place of q_{c}(x) to estimate Equation 1 via Monte Carlo sampling without the need of building a nearest neighbor graph. This result could be also used to guide the definition of new graph construction strategies based on attribute similarity criteria, for instance by proposing a good E for the data at hand.

In some embodiments, the properties of the embedding space created by deep graph networks under the k-nearest neighbor graph is investigated. The goal is to understand whether using such a graph can improve the separability between samples belonging to different classes or not. Provided that the assumptions hold, both conclusions would be interesting: if the k-nearest neighbor graph helps, then the conditions for that to happen are known; if that is not the case, the need for new graph construction mechanisms is identified and formalized.

Akin to previous methods, a 1-layer deep graph network is considered with the following neighborhood aggregation scheme that computes node embeddings hᵤ∈ [symbol] ^{D}∀u∈ [symbol] :

$${h}_{u} = \frac{1}{k} {\sum }_{v ∈ {𝒩}_{u}} {x}_{v} . \qquad ( 9 )$$

The node embedding of sample u corresponding is then fed into a standard machine learning classifier, e.g., an MLP. As done in previous works, it is assume that a linear (learnable) transformation W∈ [symbol] ^{D×D} of the input xᵥ, often used in deep graph network models such as the neural network for graphs and the graph convolutional network, is absorbed by the subsequent classifier.

Mimicking the behavior of the k-nearest neighbor algorithm, which connects similar entities together, the attribute distribution of a neighbor v∈ [symbol] is modelled as a normal distribution [symbol] (xᵥ;xᵤ,diag(σ², . . . , σ²), where σ² is a hyper-parameter that ensures it is highly unlikely to sample neighbors outside of H_{ε}(xᵤ); from now on the symbol σ_{ε} is used to make this connection clear. Under the assumptions, neighbors' sampling is repeated k times and the attributes are averaged together. Therefore, the statistical properties of normal distributions are used to compute the resulting node u's embedding distribution:

$$p ( {h}_{u} \vert {x}_{u} ) = 𝒩 ( {h}_{u} ; {x}_{u} , {Λ}_{ε} ) , {Λ}_{ε} = diag ( {σ}_{ε}^{2} / k , … , {σ}_{ε}^{2} / k ) . \qquad ( 10 )$$

Intuitively, the more neighbors a node has the more skewed the resulting distribution is around xᵤ, which makes sense if the k-nearest neighbor algorithm is applied to an infinitely large dataset.

To understand how Equation 9 affects the separability of samples belonging to different classes, a divergence score between the distributions p(h|c) and p(h|c′) is computed. When this divergence is higher than that of the distributions p(x|c) and p(x|c′), then the k-nearest neighbor structure and the inductive bias of deep graph networks are helpful for the task. Below, it is shown that for two mixtures of Gaussians the analytical form of their Squared Error Distance (SED) is obtained, the simplest symmetric divergence. This provides a concrete strategy to understand, regardless of training, if it would make sense to build a k-nearest neighbor graph for the problem. In some embodiments, this also makes a simple but meaningful connection to the over-smoothing problem.

When considering the data model 302 of FIG. 3A, it is assumed that the entity embeddings of a 1-layer deep graph network are applied on an artificially built k-nearest neighbor graph follow the distribution of Equation 10. Then the Squared Error Distances SED(p(h|c),p(h|c′)) and SED(p(x|c),p(x|c′)) have analytical forms.

As an immediate but fundamental corollary, a k-nearest neighbor graph improves the ability to distinguish samples of different classes c, c′ if it holds that SED(p(h|c),p(h|c′))>SED(p(x|c),p(x|c′)). Indeed, if class distributions diverge more in the embedding space, which has the same dimensionality as the input space, then they can be easier to separate by a universal approximator such as an MLP. This corollary is used in experiments, by reverse-engineering to find out “good” data models.

Embodiments of the present invention provide for more accurate estimates of the distributions of interest. In some embodiments, a hierarchical Naïve Bayes assumption is considered for the data model 302 of FIG. 3A, meaning that attributes are assumed to be independent when conditioned on a specific mixture and class. This might not be the case in real-world data, so it would be useful to consider dependent attributes as well. Second, the analysis has considered hyper-cubes in the interest of simplicity, but nearest neighbor graphs sometimes consider hyper-balls centered at a point. In high-dimensional spaces, the volume of hyper-cubes and hyper-balls can differ in non-trivial ways, so future work could consider extending the results to hyper-balls. The data model also assumes that the conditionally independent attribute distributions share the same mixture weights, but a more general model would assume that each attribute is described by an attribute-specific mixture of distributions. This would allow to better model, for instance, categorical attributes. It is already possible to extend some of the results to this scenario since a product of mixtures is still a mixture. However, the resulting number of mixtures would be exponential in the number of mixture components and the computational complexity quickly becomes impractical for the empirical analysis. Finally, as done in previous works, the non-linearity in the neighborhood aggregation of Equation 9 is ignored. As is observed, despite this simplification the intuitions that have been gained from the analysis seem to be reflected in the empirical results.

The analysis studies the impact of artificial structures in graph representation learning and enables to warn machine learning practitioners or systems about the potential downsides of certain nearest neighbor strategies.

In some embodiments, quantitative and qualitative experiments were conducted to support the theoretical insights. For the experiments, a server with 32 cores, 128 GBs of RAM, and 4 GPUs with 11 GBs of memory was used.

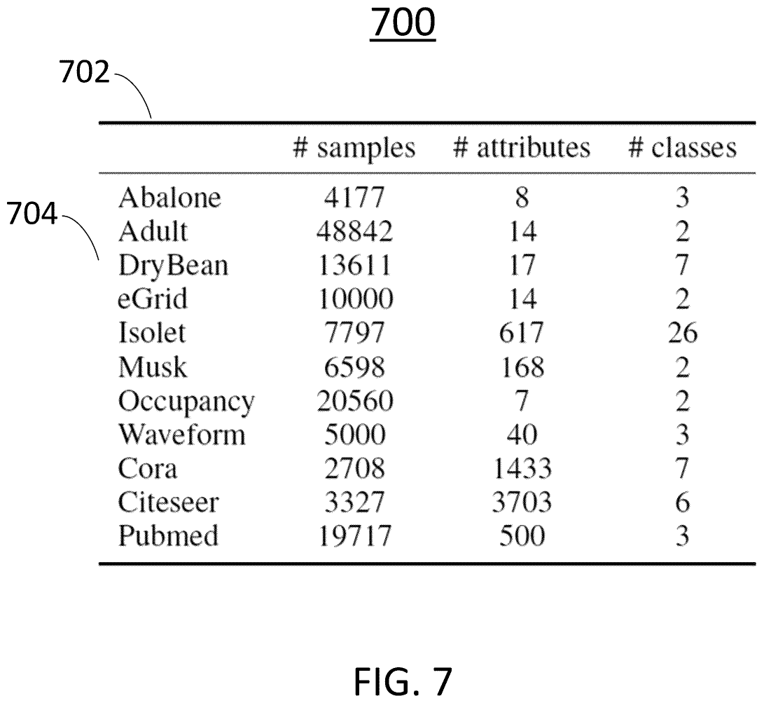

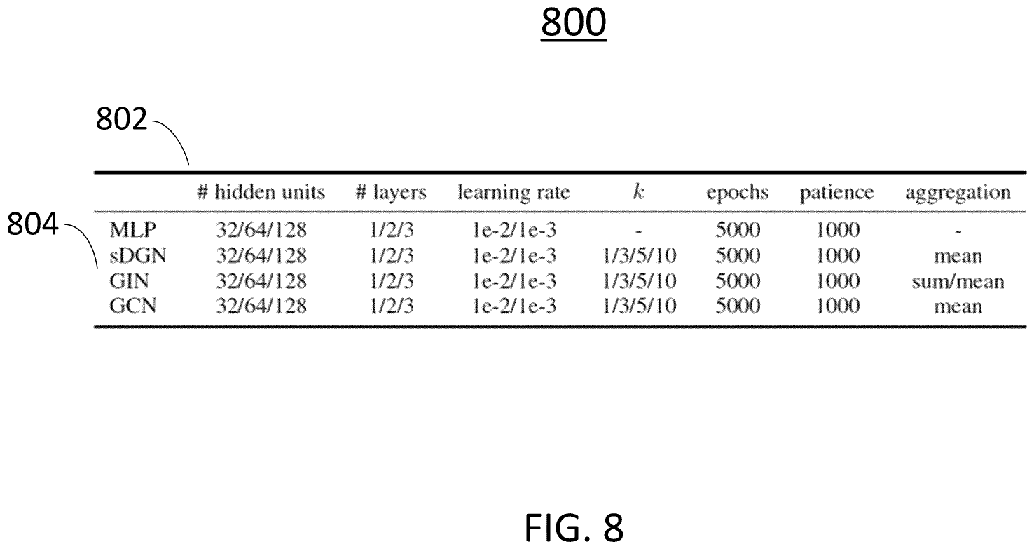

Quantitatively speaking, a structure-agnostic baseline is compared against different graph machine learning models, namely a simple deep graph network that implements Equation 9 followed by a multi-layer perceptron (MLP) classifier, the graph isomorphism network and the graph convolutional network. The goal is to show that when all training labels are available, using a k-nearest neighbor graph does not offer any concrete benefit. 11 datasets are considered, eight of which were taken from a repository, namely Abalone, Adult, Dry Bean, Electrical Grid Stability, Isolet, Musk v2, Occupancy Detection, Waveform Database Generator v2, as well as the citation networks Cora, Citeseer, and Pubmed. For each dataset, a k-nearest neighbor graph is built, where k is a hyper-parameter, using the node attributes' similarity to find neighbors (discarding the original structure in the citation networks). In some embodiments, some of the datasets statistics are depicted in table 700 of FIG. 7. For every dataset and model, a rigorous and fair evaluation setup of is followed: a 0-fold cross validation for risk assessment is performed, with a hold-out model selection (90% training/10% validation) for each of the 10 external training folds. the hyper-parameters for the model selection phase are reported in table 800 of FIG. 8. For each of the 10 best models, 3 final re-training runs are performed and their test performances are averaged to mitigate bad initializations.

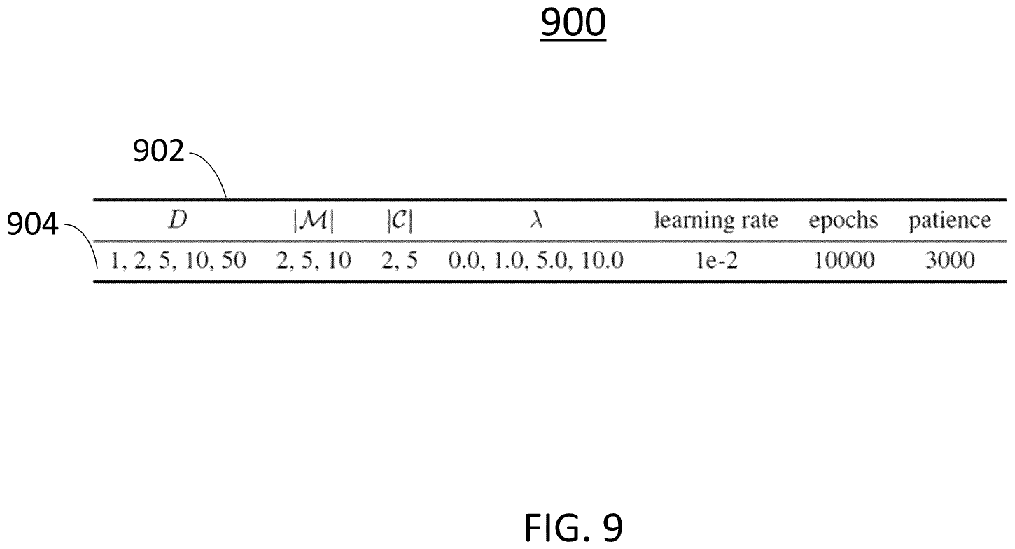

Two qualitative experiments are provided. First, the theoretical results are reverse-engineered to find, if possible, data distributions satisfying SED(p(h|c),p(h|c′))>SED(p(x|c),p(x|c′)). There is no dataset associated with this experiment, rather it is learned that the parameters of the graphical model of data model 302 of FIG. 3A together with ε,k,σ_{ε} (k is treated as a continuous value during the optimization) in order to maximize the following objective: [symbol] _{SED}−λ [symbol] _{CCNS} where λ is a hyper-parameter. [symbol] _{SED} sums the above inequality for all pairs of distinct classes, whereas [symbol] _{CCNS} computes the lower bound for the inter-class similarity, thus acting as a regularizer that avoids the trivial solution p(x|c)=p(x|c′) for class pairs c, c′. The set of configurations for this experiment is reported in table 900 of FIG. 9, and the Adam optimizer is used.

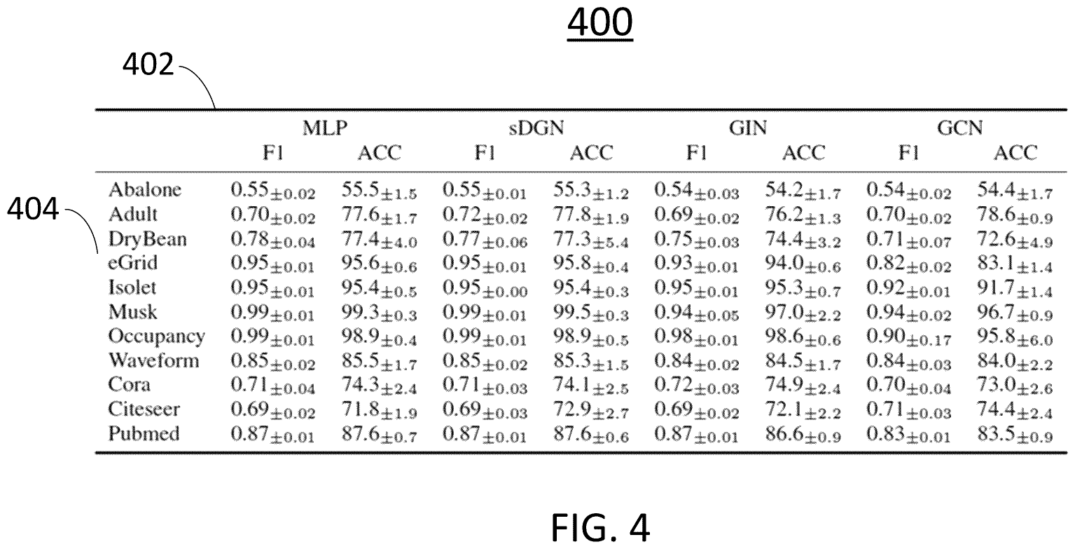

Table 400 of FIG. 4 depicts node classification mean and standard deviation results for a structure-agnostic baseline (MLP) compared to graph machine learning methods applied to artificially built k-nearest neighbor graphs. Model selection w.r.t. the F1 score was performed, but the accuracy is included for completeness. For example, k-nearest neighbor graphs are built for each of the 11 datasets as shown in column 404 of table 400. For each of the k-nearest neighbor graph of each dataset, mean and standard deviation results are computed for different machine learning models, such as MLP, simple deep graph network, graph isomorphism networks and graph convolutional networks. F1 and accuracy scores are computed, and the selection of the model is done based on the F1 score. The selection of the k is also part of the selection of the model. The pair of k and the model configuration that gives the best results is selected.

In the second qualitative experiment, the true cross-class neighborhood similarity is computed and its approximations for the specific example of FIG. 3B, show that the quantities obtained can be good representatives of the cross-class neighborhood similarity for nearest neighbor graphs.

In some embodiments, empirical results are presented to demonstrate technical and computational improvements. Statistics on the chosen hyper-parameters and additional ablation studies are shown below.

FIG. 4 details the empirical comparison between the structure-agnostic MLP and the structure-aware baselines sDGN, GIN, and GCN. In terms of micro F1 score which is the metric all models are optimized against, it is observed that there is no statistically significant improvement in performance for the different DGNs when using an artificial k-nearest neighbor graph. When looking at accuracy one can observe a similar trend, but the results fluctuate more because accuracy is not the reference metric. These results further confirm that using k-nearest neighbor graphs does not bring a substantial contribution to the generalization performances. On the contrary, it would seem the k-nearest neighbor graph can even be considered harmful for the performances, e.g., DryBean, Musk, eGrid, Waveform. It is hypothesized that the information flow of message passing does not act anymore as a regularizer for the scarce supervision scenario, rather it starts to introduce too much noise in the learning process. This is possibly the reason why simple deep graph network, which is also the simplest of the deep graph networks considered, tends to overfit less such noise and always performs on par with the MLP.

Therefore, one of the main takeaways of the quantitative analysis is that the k-nearest neighbor graph might generally not be a good artificial structure for addressing the classification of tabular data using graph representation learning methods. Different artificial structures can be proposed to create graphs that help deep graph networks to better generalize in this context. It is noted that this research direction is broader than tabular classification, but tabular data offers a great starting point since samples are assumed to be identically independently distributed and are thus easier to manipulate.

These results also substantiate the conjecture that the k-nearest neighbor graph never induces a better class separability in the embedding space. Since p(h|c) is a mixture of distributions with the same mean but higher variance than p(x|c), in a low-dimensional space intuition suggests that the distributions of different classes can always overlap more in the embedding space than in the input space.

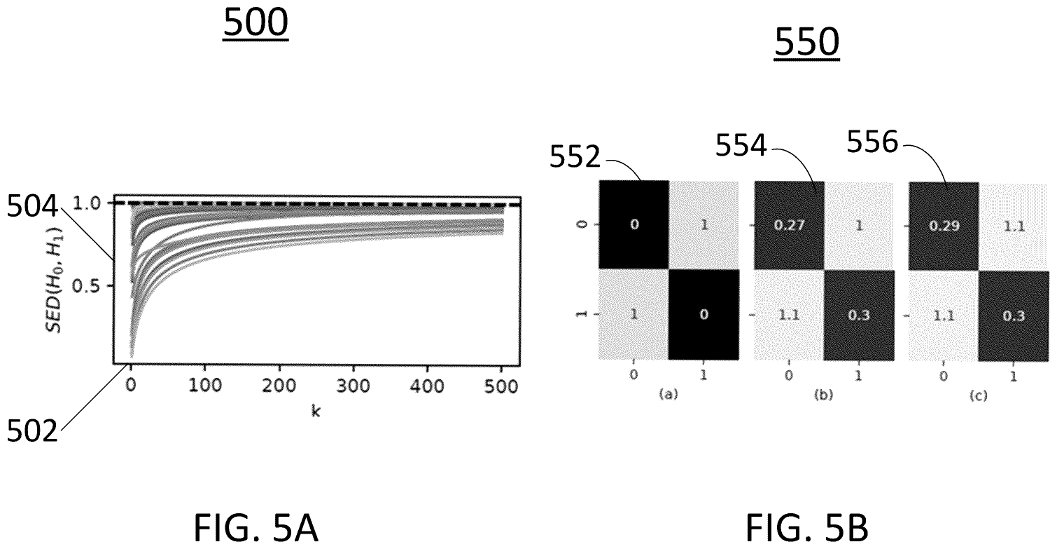

FIG. 5A depicts a graph 500 with various curves, where each curve specifies the variation of SED(p(h|c),p(h|c′)) on the y-axis against different values of k on the x-axis, for different hyperparameters' configurations that have been trained to maximize [symbol] _{SED}. The curves are normalized w.r.t their corresponding SED(p(x|c),p(x|c′)).

FIG. 5B depicts heatmaps 552, 554, and 556. The cross-class neighborhood similarity lower bound 552, its Monte Carlo approximation for ε≈0.1, obtained without creating any graph 554, and the true cross-class neighborhood similarity 556 for the example of FIGS. 3A and 3B and a 5-nearest neighbor graph.

A value in the CCNS indicates the Euclidean distance (expected/approximated for the first two matrices, and true for the third one) between each pair of classes in terms of neighborhood class (dis)similarity.

The first matrix on the left 552 represents a lower bound of the CCNS matrix computed by the theoretical framework. The matrix at the center 554 corresponds to the approximated CCNS using the framework, computed by applying Monte Carlo sampling. The matrix on the right 556 is the “true” CCNS, which is obtained by assuming that the true graph was a 5-nearest neighbor graph.

The qualitative results are presented in FIG. 5A. For each possible configuration of the models tried in table 400 of FIG. 4, the [symbol] _{SED} value is computed for varying values of k∈[1, 500], to show that the above intuition seems to hold. The values are normalized for readability, by dividing each value of SED(p(h|c),p(h|c′)) by SED(p(x|c),p(x|c′)), as the latter is independent of k. This particular figure depicts the curves for the binary classification cases, but the conclusions do not change for multi-class classification, e.g., with 5 classes.

The normalized value of SED (p(h|c),p(h|c′)) plotted on the y-axis of table 400 is always upper-bounded by 1, meaning that SED(p(h|c),p(h|c′))<SED(p(x|c),p(x|c′)) for all the configurations tried, even in higher dimensions. This result indicates that it could be unlikely to deal with a real-world dataset where a k-nearest neighbor graph induces better class separability.

Lastly, the data distribution of FIG. 3B shows how to approximate the true cross-class neighborhood similarity under the k-nearest neighbor graph with the theoretical framework. A 10000 data points were generated and k=5 was chosen to connect them. The lower bound of the cross-class neighborhood similarity is computed in the first heatmap 552, a Monte Carlo (MC) approximation of Equation 1 (1000 samples per class pair) in the heatmap 554, and the true cross-class neighborhood similarity in the heatmap 556. In this case the lower bound has computed a good approximation of the inter-class cross-class neighborhood similarity, and the Monte Carlo estimate is very close to the true cross-class neighborhood similarity. It is shown that higher values of k improve the Monte Carlo approximation. It is important to recall that both heatmaps 552 and 554 are computed using the theoretical results and an E close to the empirical one, hence it seems that the theoretical results are in accord with the evidence.

A new theoretical tool is introduced to understand how much nearest neighbor graphs are useful for the classification of tabular data. The tool and empirical evidence suggest that some attribute-based graph construction mechanisms, i.e., the k-nearest neighbor algorithm, are not a promising strategy to obtain better generalization performances. This is a particularly troubling result since the k-NN graph has been often used in the literature when a graph structure was not available. It is argued that great care should be used in the future in the empirical evaluations of such techniques, and it is recommended to always make a comparison with structure-agnostic baselines to ascertain real improvements from fictitious ones. Moreover, a theoretically principled way to model the cross-class neighborhood similarity is provided for nearest neighbor graphs, showing its approximation in a practical example. The results in this work can foster better strategies for the construction of artificial graphs or as a building block for new structure learning methods.

FIG. 7 depicts a table 700 that shows details of the various datasets that are used. Column 704 depicts the 11 datasets that are used. And row 702 depicts the characteristics related to the datasets. For example, columns 702 of table 700 depict information such as number of samples, attributes, and classes related to each dataset depicted in column 704.

The table 800 of FIG. 8 specifies the configurations tried by the model selection procedure for all the baselines considered in this work. A patience-based early stopping technique is applied on the validation micro F1-score as the only regularization technique. The free parameters' budget of all three baselines is the same. Adam was used to train all models. For example, in graph convolutional network, the use of the normalized Laplacian implies a mean aggregation.

The table 900 of FIG. 9, instead, reports the configurations tried during the qualitative experiments. A patience-based early stopping technique is applied on [symbol] _{SED}.

Equation 3 has the following analytical form

$${M}_{x} ( c ) = p ( c ) {\sum }_{m}^{\vert ℳ \vert} p ( m \vert c ) {\prod }_{f = 1}^{D} ( {F}_{{Z}_{cmf}} ( {x}_{f} + \frac{ε}{2} ) - {F}_{{Z}_{cmf}} ( {x}_{f} - \frac{ε}{2} ) ) \qquad ( 11 )$$

where Z_{cmf} is a random variables with Gaussian distribution p(w|m,c)= [symbol] (w;μ_{cmf},σ_{cmf}²).

Proof. When features are independent, one can compute the integral of a product as a product of integrals over the independent dimensions. Defining

$${a}_{f} = {x}_{f} - \frac{ε}{2} and {b}_{f} = {x}_{f} + \frac{ε}{2} ,$$

it is seen that

$${𝔼}_{w ∈ {H}_{ε} ( x )} [ p ( C = c \vert w ) ] = p ( c ) {\int }_{w ∈ {H}_{ε} ( x )} p ( w \vert c ) dw \qquad ( 12 )$$

$$= p ( c ) {\int }_{w ∈ {H}_{ε} ( x )} {\sum }_{m}^{\vert ℳ \vert} p ( w \vert m , c ) p ( m \vert c ) dw \qquad ( 13 )$$

$$= p ( c ) {\sum }_{m}^{\vert ℳ \vert} p ( m \vert c ) {\int }_{w ∈ {H}_{ε} ( x )} p ( w \vert m , c ) dw \qquad ( 14 )$$

$$= p ( c ) {\sum }_{m}^{\vert ℳ \vert} p ( m \vert c ) {\int }_{{a}_{1}}^{{b}_{1}} … {\int }_{{a}_{D}}^{{b}_{D}} {\prod }_{f = 1}^{D} p ( {w}_{f} \vert m , c ) {dw}_{D} … {dw}_{1} \qquad ( 15 )$$

$$= p ( c ) {\sum }_{m}^{\vert ℳ \vert} p ( m \vert c ) {\prod }_{f = 1}^{D} {\int }_{{a}_{f}}^{{b}_{f}} p ( {w}_{f} \vert m , c ) {dw}_{f} \qquad ( 16 )$$

$$= p ( c ) {\sum }_{m}^{\vert ℳ \vert} p ( m \vert c ) {\prod }_{f = 1}^{D} ( {F}_{{Z}_{cmf}} ( {x}_{f} + \frac{ε}{2} ) - {F}_{{Z}_{cmf}} ( {x}_{f} - \frac{ε}{2} ) ) , \qquad ( 17 )$$

where Z_{cmf}˜p(w|m,c)= [symbol] (w;μ_{cmf},σ_{cmf}²) and the last equality follows from the known fact p(a≤X≤b)=F(b)−F(a).

The following lemmas are about addition, linear transformation, and marginalization involving mixtures of distributions and are useful in the proofs.

Lemma A.1. Let X, Y be two independent random variables with corresponding mixture distributions ϕ_{X}(w)=Σᵢ^{I}αᵢfᵢ(w) and ϕ_{Y}(w)=Σⱼ^{J}βⱼgⱼ(w). Then Z=X+Y still follows a mixture distribution.

Proof. By linearity of expectation the moment generating function of X (and analogously Y) has the following form

$${M}_{X} ( t ) = 𝔼 [ {e}^{{t}^{T} w} ] = \int {e}^{{t}^{T} w} {ϕ}_{X} ( w ) dw \qquad ( 18 )$$

$$= \int {e}^{{t}^{T} w} {\sum }_{i = 1}^{I} {α}_{i} {f}_{i} ( w ) dw \qquad ( 19 )$$

$$= {\sum }_{i}^{I} {α}_{i} {M}_{{X}_{i}} ( t ) \qquad ( 20 )$$

where Xᵢ is the random variables corresponding to a component of the distribution. Using the fact that the moment generating function of Z=X+Y is given by M_{Z}(t)=M_{X}(t)M_{Y}(t), it is seen that

$${M}_{X} ( t ) = ( {\sum }_{i}^{I} {α}_{i} {M}_{{X}_{i}} ( t ) ) ( {\sum }_{j}^{J} {β}_{j} {M}_{{Y}_{j}} ( t ) ) = {\sum }_{i = 1}^{I} {\sum }_{j = 1}^{J} {α}_{i} {β}_{j} {\underbrace{{M}_{{X}_{i}} ( t ) {M}_{{Y}_{j}} ( t )}}_{{Z}_{i j} = {X}_{i} + {Y}_{j}} \qquad ( 21 )$$

Therefore, Z follows a mixture model with IJ components where each component follows the distribution associated with the random variable Zᵢⱼ=Xᵢ+Yⱼ.

Lemma A.2. Let X be a random variables with multivariate Gaussian mixture distribution ϕ_{X}(w)=Σᵢ^{I}αᵢ [symbol] (w;μᵢ,Σᵢ),w∈ [symbol] ^{D}. Then Z=ΛX, Λ∈ [symbol] ^{D×D} still follows a mixture distribution.

Proof. Using the change of variable for z=Λz it is seen that

$${ϕ}_{Z} ( w ) = {ϕ}_{X} ( {Λ}^{- 1} w ) \vert det ( {Λ}^{- 1} ) \vert = {\sum }_{i}^{I} {α}_{i} 𝒩 ( {Λ}^{- 1} w ; {μ}_{i} , {Σ}_{i} ) \vert det ( {Λ}^{- 1} ) \vert . \qquad ( 22 )$$

By expanding the terms, it is seen that the distribution of Z is still a mixture of distributions of the following form

$${ϕ}_{Z} ( w ) = {\sum }_{i}^{I} {α}_{i} 𝒩 ( w ; Λ {μ}_{i} , {ΛΣ}_{i} {Λ}^{T} ) . \qquad ( 23 )$$

Lemma A.3. Let X, Y be two independent random variables with corresponding Gaussian mixtures of distributions ϕ_{X}(w)=Σᵢ^{I}αᵢ [symbol] (w;μ_{X}_{ᵢ},σ_{X}_{ᵢ}²) and ϕ_{Y}(w)=Σⱼ^{J}βⱼ [symbol] (w;μ_{Y}_{ⱼ},σ_{Y}_{ⱼ}²). Then

$${𝔼}_{X} [ {F}_{Y} ( w ) ] = \int {F}_{Y} ( w ) {ϕ}_{X} ( w ) d w = {\sum }_{i}^{I} {\sum }_{j}^{J} {α}_{i} {β}_{j} Φ ( \frac{{μ}_{{X}_{i} -} {μ}_{{Y}_{j}}}{\sqrt{{σ}_{{Y}_{j}}^{2} + {σ}_{{X}_{i}}^{2}}} ) \qquad ( 24 )$$

Proof. It is useful to look at this integral from a probabilistic point of view. For example, it is known that

$$p ( Y \leq X \vert X = w ) = p ( Y \leq w ) = {F}_{Y} ( w ) \qquad ( 25 )$$

and that, by marginalizing over all possible values of X,

$$p ( Y \leq X ) = \int p ( Y \leq X \vert X = w ) p ( X = w ) dw = {\underbrace{\int {F}_{Y} ( w ) {ϕ}_{X} ( w ) dw .}}_{{𝔼}_{X} [ {F}_{Y} ( w ) ]} \qquad ( 26 )$$

Therefore, finding the solution corresponds to computing p(Y−X≤0). Because X, Y are independent, the resulting variable Z=Y−X is distributed as (using Lemma A.3 and Lemma A.2)

$${p}_{Z} ( w ) = {\sum }_{i}^{I} {\sum }_{j}^{J} {α}_{i} {β}_{j} 𝒩 ( w ; {μ}_{{Y}_{j}} - {μ}_{{X}_{i}} ; {σ}_{{Y}_{j}}^{2} + {σ}_{{X}_{i}}^{2} ) \qquad ( 27 )$$

and hence, using the fact that the c.d.f. of a mixture of Gaussians is the weighted sum of the individual components' c.d.f.s:

$$p ( Z \leq 0 ) = {\sum }_{i}^{I} {\sum }_{j}^{J} {α}_{i} {β}_{j} Φ ( \frac{{μ}_{{X}_{i} -} {μ}_{{Y}_{j}}}{\sqrt{{σ}_{{Y}_{j}}^{2} + {σ}_{{X}_{i}}^{2}}} ) . \qquad ( 28 )$$

Theorem 3.5. Equation 5 has the following analytical form

$${M}_{{c}^{′}} ( c ) = p ( c ) {\sum }_{m , {m}^{′}}^{\vert ℳ \vert} p ( {m}^{′} \vert {c}^{′} ) p ( m \vert c ) {\prod }_{f = 1}^{D} ( Φ ( \frac{{μ}_{{c}^{′} {m}^{′} f} + \frac{ε}{2} - {μ}_{c m f}}{\sqrt{{σ}_{c m f}^{2} + {σ}_{{c}^{′} {m}^{′} f}^{2}}} ) - Φ ( \frac{{μ}_{{c}^{′} {m}^{′} f} - \frac{ε}{2} - {μ}_{c m f}}{\sqrt{{σ}_{c m f}^{2} + {σ}_{{c}^{′} {m}^{′} f}^{2}}} ) ) . \qquad ( 29 )$$

Proof. The formula is expanded and using the result of Proposition 3.3. The random variables are defined as Z_{imf}˜ [symbol] (w;μ_{imf},σ_{imf}²),i∈ [symbol] to write

$${𝔼}_{x \sim p ( x \vert {c}^{′} )} [ {M}_{x} ( c ) ] \qquad ( 30 )$$

$$= \int p ( c ) {\sum }_{m}^{\vert ℳ \vert} p ( m \vert c ) {\prod }_{f = 1}^{D} ( {F}_{{Z}_{cmf}} ( {x}_{f} + \frac{ε}{2} ) - {F}_{{Z}_{cmf}} ( {x}_{f} - \frac{ε}{2} ) ) p ( x \vert {c}^{′} ) dx \qquad ( 31 )$$

$$= p ( c ) \int ( {\sum }_{m}^{\vert ℳ \vert} p ( m \vert c ) {\prod }_{f = 1}^{D} ( {F}_{{Z}_{cmf}} ( {x}_{f} + \frac{ε}{2} ) - {F}_{{Z}_{c m f}} ( {x}_{f} - \frac{ε}{2} ) ) ) ( {\sum }_{{m}^{′}}^{\vert ℳ \vert} p ( x \vert {m}^{′} , {c}^{′} ) p ( {m}^{′} \vert {c}^{′} ) ) dx \qquad ( 32 )$$

$$= p ( c ) \int {\sum }_{m , {m}^{′}}^{\vert ℳ \vert} p ( {m}^{′} \vert {c}^{′} ) p ( m \vert c ) {\prod }_{f = 1}^{D} ( {F}_{{Z}_{cmf}} ( {x}_{f} + \frac{ε}{2} ) - {F}_{{Z}_{c m f}} ( {x}_{f} - \frac{ε}{2} ) ) p ( x \vert {m}^{′} , {c}^{′} ) dx \qquad ( 33 )$$

$$= p ( c ) {\sum }_{m , {m}^{′}}^{\vert ℳ \vert} p ( {m}^{′} \vert {c}^{′} ) p ( m \vert c ) \int {\prod }_{f = 1}^{D} ( {F}_{{Z}_{c m f}} ( {x}_{f} + \frac{ε}{2} ) - {F}_{{Z}_{c m f}} ( {x}_{f} - \frac{ε}{2} ) ) {\prod }_{f = 1}^{D} p ( {x}_{f} \vert {m}^{′} , {c}^{′} ) dx \qquad ( 34 )$$

$$= p ( c ) {\sum }_{m , {m}^{′}}^{\vert ℳ \vert} p ( {m}^{′} \vert {c}^{′} ) p ( m \vert c ) \int \cdot \int {\prod }_{f = 1}^{D} ( {F}_{{Z}_{c m f}} ( {x}_{f} + \frac{ε}{2} ) - {F}_{{Z}_{c m f}} ( {x}_{f} - \frac{ε}{2} ) ) p ( {x}_{f} \vert {m}^{′} , {c}^{′} ) {dx}_{D} … {dx}_{1} \qquad ( 35 )$$

$$= p ( c ) {\sum }_{m , {m}^{′}}^{\vert ℳ \vert} p ( {m}^{′} \vert {c}^{′} ) p ( m \vert c ) {\prod }_{f = 1}^{D} \int ( {F}_{{Z}_{c m f}} ( {x}_{f} + \frac{ε}{2} ) - {F}_{{Z}_{c m f}} ( {x}_{f} - \frac{ε}{2} ) ) p ( {x}_{f} \vert {c}^{′} ) {dx}_{f} \qquad ( 36 )$$

$$= p ( c ) {\sum }_{m , {m}^{′}}^{\vert ℳ \vert} p ( {m}^{′} \vert {c}^{′} ) p ( m \vert c ) {\prod }_{f = 1}^{D} {\underbrace{\int ( {F}_{{Z}_{c m f}} ( {x}_{f} + \frac{ε}{2} ) p ( {x}_{f} \vert {c}^{′} ) {dx}_{f}}}_{{𝔼}_{{Z}_{{c}_{f}^{′}}} [ {F}_{{Z}_{c m f}} ( {x}_{f} + \frac{ε}{2} ) ]} - {\underbrace{\int ( {F}_{{Z}_{c m f}} ( {x}_{f} - \frac{ε}{2} ) p ( {x}_{f} \vert {c}^{′} ) {dx}_{f}}}_{{𝔼}_{{Z}_{{c}_{f}^{′}}} [ {F}_{{Z}_{c m f}} ( {x}_{f} - \frac{ε}{2} ) ]} \qquad ( 37 )$$

It is noted that

$${F}_{{Z}_{c m f}} ( {x}_{f} + \frac{ε}{2} ) = {F}_{Y} ( {x}_{f} )$$

where Y follows distribution

$$𝒩 ( w ; {μ}_{cmf} - \frac{ε}{2} , {σ}_{cmf}^{2} )$$

(and symmetrically for

$${F}_{{Z}_{c m f}} ( {x}_{f} - \frac{ε}{2} ) ) ,$$

so Lemma A.3 can be applied and obtain

$$= p ( c ) {\sum }_{m , {m}^{′}}^{\vert ℳ \vert} p ( {m}^{′} \vert {c}^{′} ) p ( m \vert c ) {\prod }_{f = 1}^{D} ( Φ ( \frac{{μ}_{{c}^{′} {m}^{′} f} + \frac{ε}{2} - {μ}_{c m f}}{\sqrt{{σ}_{c m f}^{2} + {σ}_{{c}^{′} {m}^{′} f}^{2}}} ) - Φ ( \frac{{μ}_{{c}^{′} {m}^{′} f} - \frac{ε}{2} - {μ}_{c m f}}{\sqrt{{σ}_{c m f}^{2} + {σ}_{{c}^{′} {m}^{′} f}^{2}}} ) ) . \qquad ( 38 )$$

Proposition 3.7. Let the first D derivatives of [symbol] M_{c′}(i) be different from 0 in an open interval I around ε=0. Then Equation 7 has the following limits (the first of which requires the assumption)

$$\begin{matrix}{lim}_{ε \to 0} {p}_{{c}^{′}} ( c ) = \frac{p ( c ) {\sum }_{m , {m}^{′}}^{\vert ℳ \vert} p ( m \vert c ) p ( {m}^{′} \vert {c}^{′} ) {\prod }_{f = 1}^{D} {ϕ}_{{Z}_{{cmm}^{′} f}} ( 0 )}{{\sum }_{i}^{\vert 𝒞 \vert} p ( i ) {\sum }_{m , {m}^{′}}^{\vert ℳ \vert} p ( m \vert c ) p ( {m}^{′} \vert {c}^{′} ) {\prod }_{f = 1}^{D} {ϕ}_{{Z}_{{imm}^{′} f}} ( 0 )} & {lim}_{ε \to ∞} {p}_{{c}^{′}} ( c ) \\ = p ( c ) & \end{matrix} \qquad ( 39 )$$

where Z_{imm′f} has distribution [symbol] (·;−σ_{imm′f},b_{imm′f}²), a_{imm′f}=²(μ_{c′m′f}−μ_{imf}) and b_{imm′f}=2\sqrt{σ_{imf}²+σ_{c′m′f}²}.

Proof. The second limit follows from lim_{x→+∞}Φ(x)=1 and lim_{x→-∞}Φ(x)=0. As regards the first limit, the terms are first expanded

$$( 40 )$$

$${lim}_{ε \to 0} {p}_{{c}^{′}} ( c ) = {lim}_{ε \to 0} \frac{\begin{matrix}p ( c ) {\sum }_{m , {m}^{′}}^{\vert ℳ \vert} p ( m \vert c ) p ( {m}^{′} \vert {c}^{′} ) {\prod }_{f = 1}^{D} ( Φ ( \frac{ε + {a}_{{cmm}^{′} f}}{{b}_{{cmm}^{′} f}} ) - \\ Φ ( \frac{- ε + {a}_{{cmm}^{′} f}}{{b}_{{cmm}^{′} f}} ) )\end{matrix}}{\begin{matrix}{\sum }_{i}^{\vert 𝒞 \vert} p ( i ) {\sum }_{m , {m}^{′}}^{\vert ℳ \vert} p ( m \vert i ) p ( {m}^{′} \vert {c}^{′} ) {\prod }_{f = 1}^{D} ( Φ ( \frac{ε + {a}_{{imm}^{′} f}}{{b}_{{imm}^{′} f}} ) - \\ Φ ( \frac{- ε + {a}_{{imm}^{′} f}}{{b}_{{imm}^{′} f}} ) )\end{matrix}} ,$$

where a_{imm′f}=2(μ_{c′m′f}−μ_{imf}) and b_{imm′f}=2\sqrt{σ_{imf}²+σ_{c′m′f}²}∀i∈ [symbol] to simplify the notation. By defining Z_{imm′f}˜ [symbol] (·;−a_{imm′f}, b_{imm′f}²), the limit can be rewritten as

$${lim}_{ε \to 0} \frac{p ( c ) {\sum }_{m , {m}^{′}}^{\vert ℳ \vert} p ( m \vert c ) p ( {m}^{′} \vert {c}^{′} ) {\prod }_{f = 1}^{D} {F}_{{Z}_{{cmm}^{′} f}} ( ε ) - {F}_{{Z}_{{cmm}^{′} f}} ( - ε )}{{\sum }_{i}^{\vert 𝒞 \vert} p ( i ) {\sum }_{m , {m}^{′}}^{\vert ℳ \vert} p ( m \vert c ) p ( {m}^{′} \vert {c}^{′} ) {\prod }_{f = 1}^{D} {F}_{{Z}_{{cmm}^{′} f}} ( ε ) - {F}_{{Z}_{{cmm}^{′} f}} ( - ε )} \qquad ( 41 )$$

$$= {lim}_{ε \to 0} \frac{p ( c ) {\sum }_{m , {m}^{′}}^{\vert ℳ \vert} p ( m \vert c ) p ( {m}^{′} \vert {c}^{′} ) {\prod }_{f = 1}^{D} {g}_{{cmm}^{′} f} ( ε )}{{\sum }_{i}^{\vert 𝒞 \vert} p ( i ) {\sum }_{m , {m}^{′}}^{\vert ℳ \vert} p ( m \vert c ) p ( {m}^{′} \vert {c}^{′} ) {\prod }_{f = 1}^{D} {g}_{{imm}^{′} f} ( ε )} , \qquad ( 42 )$$

Both the numerator and denominator tend to zero in the limit, so L'Hôpital's rule, is applied, potentially multiple times.

For reasons that can become clear soon, it is shown that the limit of the first derivative of each term in the product is not zero nor infinity:

$${lim}_{ε \to 0} {g}_{{imm}^{′} f}^{′} ( ε ) = {lim}_{ε \to 0} {ϕ}_{{Z}_{{imm}^{′} f}} ( ε ) + {ϕ}_{{Z}_{{imm}^{′} f}} ( - ε ) = 2 {ϕ}_{{Z}_{{imm}^{′} f}} ( 0 ) , \qquad ( 43 )$$

In addition, let us consider the n-th derivative of the product of functions (by applying the generalized product rule):

$${( {\prod }_{f = 1}^{D} {g}_{{imm}^{′} f} ( ε ) )}^{( n )} = {\sum }_{{j}_{1} + … + {j}_{D} = n} ( \begin{matrix}n \\ {j}_{1} , … , {j}_{D}\end{matrix} ) {\prod }_{f = 1}^{D} {g}_{{imm}^{′} f}^{( {j}_{f} )} ( ε ) \qquad ( 44 )$$

and it is noted that, as long as n<D there can exist an assignment to j_{f}=0 ∀f in the summation, and thus the limit of each inner product can always tend to 0 when E goes to 0. However, when the D-th derivative is taken, there exists one term in the summation that does not go to 0 in the limit, which is

$$( \begin{matrix}D \\ 1 , … , 1\end{matrix} ) {\prod }_{f = 1}^{D} {g}_{{imm}^{′} f}^{′} ( ε ) = D ! {\prod }_{f = 1}^{D} {g}_{{imm}^{′} f}^{′} ( ε ) \qquad ( 45 )$$

$${lim}_{ε \to 0} ( \begin{matrix}D \\ 1 , … , 1\end{matrix} ) {\prod }_{f = 1}^{D} {g}_{{imm}^{′} f}^{′} ( ε ) = {2}^{D} D ! {\prod }_{f = 1}^{D} {ϕ}_{{Z}_{{imm}^{′} f}} ( 0 ) ( ε ) \qquad ( 46 )$$

Therefore, by applying the L'Hôpital's rule D times:

$${lim}_{ε \to 0} \frac{p ( c ) {\sum }_{m , {m}^{′}}^{\vert ℳ \vert} p ( m \vert c ) p ( {m}^{′} \vert {c}^{′} ) {\prod }_{f = 1}^{D} {g}_{{cmm}^{′} f} ( ε )}{{\sum }_{i}^{\vert 𝒞 \vert} p ( i ) {\sum }_{m , {m}^{′}}^{\vert ℳ \vert} p ( m \vert c ) p ( {m}^{′} \vert {c}^{′} ) {\prod }_{f = 1}^{D} {g}_{{imm}^{′} f} ( ε )} \qquad ( 47 )$$

$$= \frac{p ( c ) {2}^{D} D ! {\sum }_{m , {m}^{′}}^{\vert ℳ \vert} p ( m \vert c ) p ( {m}^{′} \vert {c}^{′} ) {\prod }_{f = 1}^{D} {ϕ}_{{Z}_{{cmm}^{′} f}} ( 0 )}{{2}^{D} D ! {\sum }_{i}^{\vert 𝒞 \vert} p ( i ) {\sum }_{m , {m}^{′}}^{\vert ℳ \vert} p ( m \vert c ) p ( {m}^{′} \vert {c}^{′} ) {\prod }_{f = 1}^{D} {ϕ}_{{Z}_{{imm}^{′} f}} ( 0 )} . \qquad ( 48 )$$

However, to be valid, L'Hôpital's rule requires that the derivative of the denominator never goes to 0 for points different from 0.

For n=1, this holds as g_{imm′f}′(ε)>0∀ε,g_{imm′f}(ε)≥0 and g_{imm′f}(ε)=0⇔ε=0. In fact,

$${( {\prod }_{f = 1}^{D} {g}_{{imm}^{′} f} ( ε ) )}^{( 1 )} = {\sum }_{{j}_{1} + … + {j}_{D} = 1} {\prod }_{f = 1}^{D} {g}_{{imm}^{′} f}^{( {j}_{f} )} ( ε ) \qquad ( 49 )$$

is a sum over terms that are all greater than 0 for ε≠0.

For 1<n≤D, the hypothesis is used to conclude the proof.

Analytical form of the n-th derivative of [symbol] M_{c′}(i) In order to verify if the hypothesis of Proposition 3.7 is true given the parameters of all features' distributions, one could compute the n-th derivative of the denominator w.r.t. E and check that it is not zero around 0. Starting from

$${( {\prod }_{f = 1}^{D} {g}_{{imm}^{′} f} ( ε ) )}^{( n )} = {\sum }_{{j}_{1} + … + {j}_{D} = n} ( \begin{matrix}n \\ {j}_{1} , … , {j}_{D}\end{matrix} ) {\prod }_{f = 1}^{D} {g}_{{imm}^{′} f}^{( {j}_{f} )} ( ε ) \qquad ( 50 )$$

and proceeding using the fact that the n-th derivative of the standard normal distribution S˜ [symbol] (0, 1) has a well-known form in terms of the n-th (probabilist) Hermite polynomial Hₑ_{ₙ}(x). The following equivalence is noted:

$${ϕ}_{{Z}_{{imm}^{′} f}} ( w ) = \frac{1}{{b}_{{imm}^{′} f}} {ϕ}_{S} ( \frac{w + {a}_{{imm}^{′} f}}{{b}_{{imm}^{′} f}} ) , \qquad ( 51 )$$

$$hence$$

$$\begin{matrix}{g}_{{imm}^{′} f}^{( n )} ( ε ) & = & {( {{g}^{′}}_{{imm}^{′} f} ( ε ) )}^{( n - 1 )} = {( {ϕ}_{{Z}_{{imm}^{′} f}} ( ε ) + {ϕ}_{{Z}_{{imm}^{′} f}} ( - ε ) )}^{( n - 1 )} & ( 52 ) \\ & = & {( {ϕ}_{{Z}_{{imm}^{′} f}} ( ε ) )}^{( n - 1 )} + {( {ϕ}_{{Z}_{{imm}^{′} f}} ( - ε ) )}^{( n - 1 )} & ( 53 ) \\ & = & {( \frac{1}{{b}_{{imm}^{′} f}} {ϕ}_{S} ( \frac{ε + {a}_{{imm}^{′} f}}{{b}_{{imm}^{′} f}} ) )}^{( n - 1 )} & ( 54 ) \\ & + & {( \frac{1}{{b}_{{imm}^{′} f}} {ϕ}_{S} ( \frac{- ε + {a}_{{imm}^{′} f}}{{b}_{{imm}^{′} f}} ) )}^{( n - 1 )} & ( 55 )\end{matrix}$$

$$= \frac{1}{{b}_{{imm}^{′} f}} ( {( {ϕ}_{S} ( \frac{ε + {a}_{{imm}^{′} f}}{{b}_{{imm}^{′} f}} ) )}^{( n - 1 )} + {( {ϕ}_{S} ( \frac{- ε + {a}_{{imm}^{′} f}}{{b}_{{imm}^{′} f}} ) )}^{( n - 1 )} ) \qquad ( 56 )$$

$$= \frac{1}{{b}_{{imm}^{′} f}} ( {( 𝒩 ( \frac{ε + {a}_{{imm}^{′} f}}{{b}_{{imm}^{′} f}} ; 0 , 1 ) )}^{( n - 1 )} + {( 𝒩 ( \frac{- ε + {a}_{{imm}^{′} f}}{{b}_{{imm}^{′} f}} ; 0 , 1 ) )}^{( n - 1 )} ) \qquad ( 57 )$$

$$+ {( - 1 )}^{n - 1} {H}_{{e}_{n - 1}} ( \frac{- ε + {a}_{{imm}^{′} f}}{{b}_{{imm}^{′} f}} ) {ϕ}_{S} ( \frac{- ε + {a}_{{imm}^{′} f}}{{b}_{{imm}^{′} f}} ) ) \qquad ( 58 )$$

This result is used in the expansion of Equation 50

$${\sum }_{{j}_{1} + … + {j}_{D} = n} ( \begin{matrix}n \\ {j}_{1} , … , {j}_{D}\end{matrix} ) {\prod }_{f = 1}^{D} {g}_{{imm}^{′} f}^{( {j}_{f} )} ( ε ) \qquad ( 59 )$$

$$= {\sum }_{{j}_{1} + … + {j}_{D} = n} ( \begin{matrix}n \\ {j}_{1} , … , {j}_{D}\end{matrix} ) {{\prod }_{{j}_{f} > 0}}_{f = 1 … D} {g}_{{imm}^{′} f}^{( {j}_{f} )} ( ε ) {{\prod }_{{j}_{f} = 0}}_{f = 1 … D} {g}_{{imm}^{′} f} ( ε ) . \qquad ( 60 )$$

However, it is readily seen that computing the derivative for a single E has combinatorial complexity, which makes the application of the above formulas practical only for small values of D.

A result is now presented that can help compute the SED divergence between two Gaussian mixtures of distributions.

Lemma A.4. Let X, Y be two independent random variables with corresponding Gaussian mixture distributions ϕ_{X}(w)=Σᵢ^{I}αᵢf_{X}_{ᵢ}(w)=Σᵢ^{I}αᵢ [symbol] (w;μᵢ^{X},Σᵢ^{X}) and ϕ_{Y}(w)=Σⱼ^{J}βⱼf_{Y}_{ⱼ}(w)=Σⱼ^{J}βⱼ [symbol] (w;μⱼ^{Y},Σⱼ^{Y}),w∈ [symbol] ^{D}. Then the SED divergence between ϕ_{X}(w) and ϕ_{Y}(w) can be computed as

$$SED ( {ϕ}_{X} , {ϕ}_{Y} ) = {\sum }_{i}^{I} {\sum }_{j}^{I} {α}_{i} {α}_{j} {A}_{i , j} + {\sum }_{i}^{J} {\sum }_{j}^{J} {β}_{i} {β}_{j} {B}_{i , j} - 2 {\sum }_{i}^{I} {\sum }_{j}^{J} {α}_{i} {β}_{j} {C}_{i , j} , \qquad ( 61 )$$

$$where \qquad ( 62 )$$

$${A}_{i , j} = 𝒩 ( {μ}_{i}^{X} \vert {μ}_{j}^{X} , ( {\sum }_{i}^{X} + {\sum }_{j}^{X} ) ) \qquad ( 63 )$$

$${B}_{i , j} = 𝒩 ( {μ}_{i}^{Y} \vert {μ}_{j}^{Y} , ( {\sum }_{i}^{Y} + {\sum }_{j}^{Y} ) ) \qquad ( 64 )$$

$${C}_{i , j} = 𝒩 ( {μ}_{i}^{X} \vert {μ}_{j}^{Y} , ( {\sum }_{i}^{X} + {\sum }_{j}^{Y} ) ) \qquad ( 65 )$$

Proof.

$$SED ( {ϕ}_{X} , {ϕ}_{Y} ) = \int {( {ϕ}_{X} ( w ) - {ϕ}_{Y} ( w ) )}^{2} dw \qquad ( 66 )$$

$$SED ( {ϕ}_{X} , {ϕ}_{Y} ) = \int {( {\sum }_{i}^{I} {α}_{i} {f}_{{X}_{i}} ( w ) - {\sum }_{j}^{J} {β}_{j} {f}_{{Y}_{j}} ( w ) )}^{2} dw \qquad ( 67 )$$

$$= {\sum }_{i}^{I} {\sum }_{j}^{I} {α}_{i} {α}_{j} \int {f}_{{X}_{i}} ( w ) {f}_{{X}_{j}} ( w ) dw + {\sum }_{i}^{J} {\sum }_{j}^{J} {β}_{i} {β}_{j} \int {f}_{{Y}_{i}} ( w ) {f}_{{Y}_{j}} ( w ) dw \qquad ( 68 )$$

$$- 2 {\sum }_{i}^{I} {\sum }_{j}^{J} {α}_{i} {β}_{j} \int {f}_{{X}_{i}} ( w ) {f}_{{Y}_{j}} ( w ) dw \qquad ( 69 )$$

Finally, the integral of the product of two Gaussians can be computed as follows

$$\int 𝒩 ( w \vert {μ}_{1} , {\sum }_{1} ) 𝒩 ( w \vert {μ}_{2} , {\sum }_{2} ) dw = 𝒩 ( {μ}_{1} \vert {μ}_{2} , ( {\sum }_{1} + {\sum }_{2} ) ) \qquad ( 70 )$$

in order to obtain the desired result.

Proposition 3.8. Consider the data model of FIG. 3A, and let us assume that the entity embeddings of a 1-layer DGN applied on an artificially built k-NN graph follow the distribution of Equation 10. Then the Squared Error Distances SED(p(h|c),p(h|c′)) and SED(p(x|c),p(x|c′)) have analytical forms.

Proof. First, it is to be shown how one can compute the conditional probabilities p(h|c) and p(h|c′), and then the analytical computation of the SED divergence can be derived to verify the inequality in a similar manner of Helén and Virtanen.

The explicit form of p(h|c) is worked out, by marginalizing out x:

$$p ( h \vert c ) = {\sum }_{m}^{\vert ℳ \vert} p ( h \vert x ) p ( x \vert m , c ) p ( m \vert c ) dx \qquad ( 71 )$$

$${\sum }_{m}^{\vert ℳ \vert} p ( m \vert c ) \int p ( h \vert x ) p ( x \vert m , c ) dx \qquad ( 72 )$$

$$= {\sum }_{m}^{\vert ℳ \vert} p ( m \vert c ) \int 𝒩 ( h ; x , {Λ}_{ε} ) 𝒩 ( x ; {μ}_{mc} , {\sum }_{mc} ) dx \qquad ( 73 )$$

$$= {\sum }_{m}^{\vert ℳ \vert} p ( m \vert c ) {\underbrace{\int 𝒩 ( h - x ; 0 , {Λ}_{ε} ) 𝒩 ( x ; {μ}_{mc} , {\sum }_{mc} ) dx}}_{Gaussian convolution} \qquad ( 74 )$$

$$= {\sum }_{m}^{\vert ℳ \vert} p ( m \vert c ) 𝒩 ( h ; {μ}_{mc} , {\sum }_{mc} + {Λ}_{ε} ) . \qquad ( 75 )$$

Therefore, the distribution resulting from the k-nearest neighbor neighborhood aggregation can change the input's variance in a way that is inversely proportional to the number of neighbors. In the limit of k→∞ and finite σ_{ε}, it follows that p(h|c)=p(x|c). Since p(h|c) still follows a mixture of distributions of known form, it is noted that a repeated aggregation of the neighborhood aggregation mechanism (without any learned transformation) would only increase the values of the covariance matrix. In turn, this would make the distribution spread more and more, causing what is known in the literature as the oversmoothing effect (see Section 2).

Since both X_{c} and H_{c} follow a Gaussian mixture distribution, Lemma A.4 is applied to obtain a closed-form solution and be able to evaluate the inequality. For example

$$\begin{matrix}SED ( {ϕ}_{{X}_{c}} , {ϕ}_{{X}_{{c}^{′}}} ) & = & & ( 76 ) \\ & = & {\sum }_{m , {m}^{′}}^{\vert ℳ \vert} p ( m \vert c ) p ( {m}^{′} \vert c ) 𝒩 ( {μ}_{cm} ; {μ}_{{cm}^{′}} , {\sum }_{cm} + {\sum }_{{cm}^{′}} ) & ( 77 ) \\ & + & {\sum }_{m , {m}^{′}}^{\vert ℳ \vert} p ( m \vert {c}^{′} ) p ( {m}^{′} \vert {c}^{′} ) 𝒩 & ( 78 ) \\ & & ( {μ}_{{c}^{′} m} ; {μ}_{{c}^{′} {m}^{′}} , {\sum }_{{c}^{′} m} + {\sum }_{{c}^{′} {m}^{′}} ) & \\ & - & 2 {\sum }_{m , {m}^{′}}^{\vert ℳ \vert} p ( m \vert c ) p ( {m}^{′} \vert {c}^{′} ) 𝒩 & ( 79 ) \\ & & ( {μ}_{cm} ; {μ}_{{c}^{′} {m}^{′}} , {\sum }_{cm} + {\sum }_{{c}^{′} {m}^{′}} ) & \end{matrix}$$

$$and$$

$$SED ( {ϕ}_{{H}_{c}} , {ϕ}_{{H}_{{c}^{′}}} ) = \qquad ( 80 )$$

$$= {\sum }_{m , {m}^{′}}^{\vert ℳ \vert} p ( m \vert c ) p ( {m}^{′} \vert c ) 𝒩 ( {μ}_{cm} ; {μ}_{{cm}^{′}} , {\sum }_{cm} + {\sum }_{{cm}^{′}} + 2 {Λ}_{ε} ) \qquad ( 81 )$$

$$+ {\sum }_{m , {m}^{′}}^{\vert ℳ \vert} p ( m \vert {c}^{′} ) p ( {m}^{′} \vert {c}^{′} ) 𝒩 ( {μ}_{{c}^{′} m} ; {μ}_{{c}^{′} {m}^{′}} , {\sum }_{{c}^{′} m} + {\sum }_{{c}^{′} {m}^{′}} + 2 {Λ}_{ε} ) \qquad ( 82 )$$

$$- 2 {\sum }_{m , {m}^{′}}^{\vert ℳ \vert} p ( m \vert c ) p ( {m}^{′} \vert {c}^{′} ) 𝒩 ( {μ}_{cm} ; {μ}_{{c}^{′} {m}^{′}} , {\sum }_{cm} + {\sum }_{{c}^{′} {m}^{′}} + 2 {Λ}_{ε} ) \qquad ( 83 )$$

Therefore, if SED(ϕ_{H}_{_{c}},ϕ_{H}_{_{c′}})>SED(ϕ_{X}_{_{c′}},ϕ_{X}_{_{c′}}), points in the deep graph network's latent space can be easier to classify than those in the input space because the former pair of distributions are more separable than the latter.

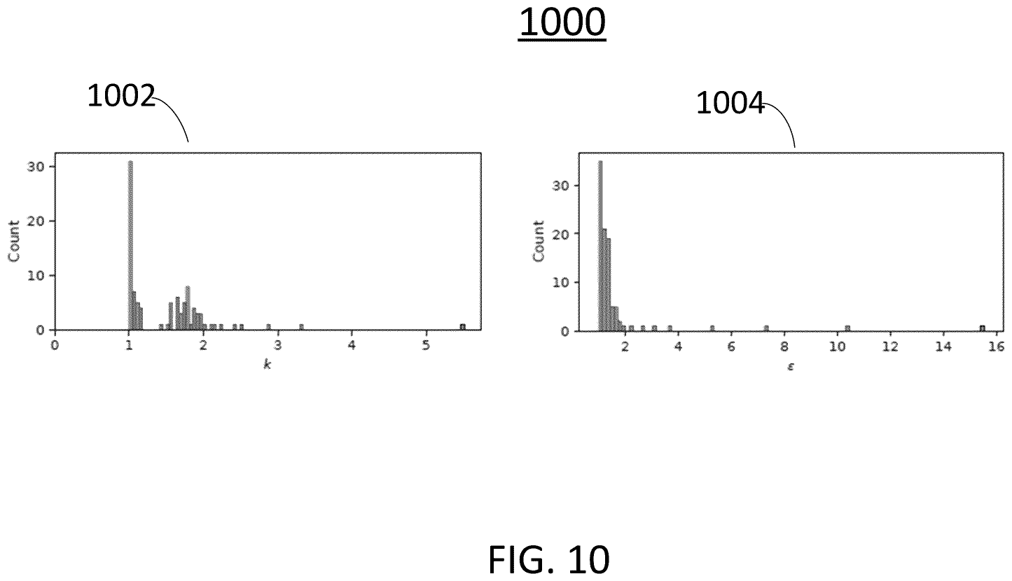

FIG. 10 illustrates histograms of the k and F chosen by each model on the different datasets during a 10-fold cross validation for risk assessment, in accordance with embodiments of the present disclosure. Graph 1002 of FIG. 10 plots the number of times a particular k value was chosen on the y-axis, and the respective k-values on the x-axis. Similarly, graph 1004 of FIG. 10 plots a count of the number of times a particular F value chosen by each model on the y-axis, and plots the corresponding F value on the x-axis.

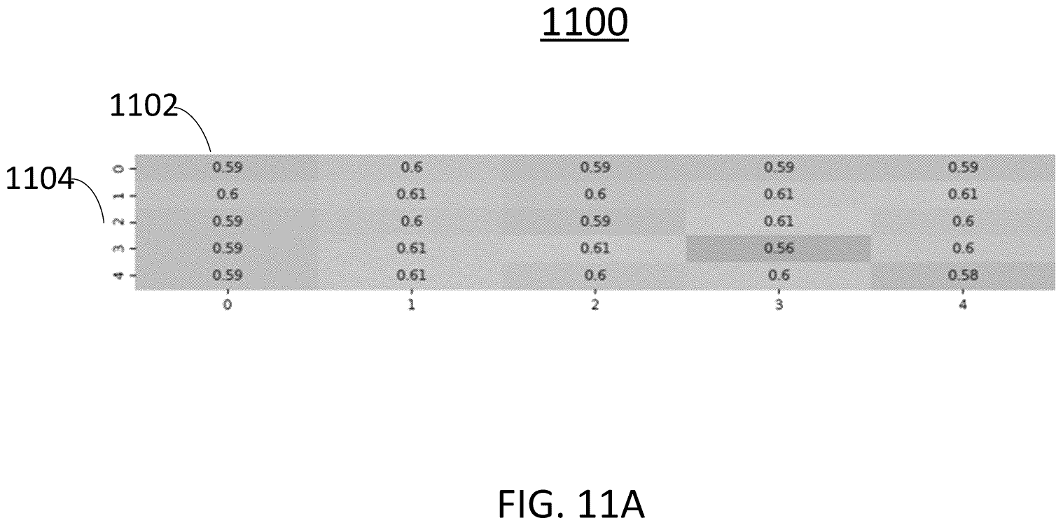

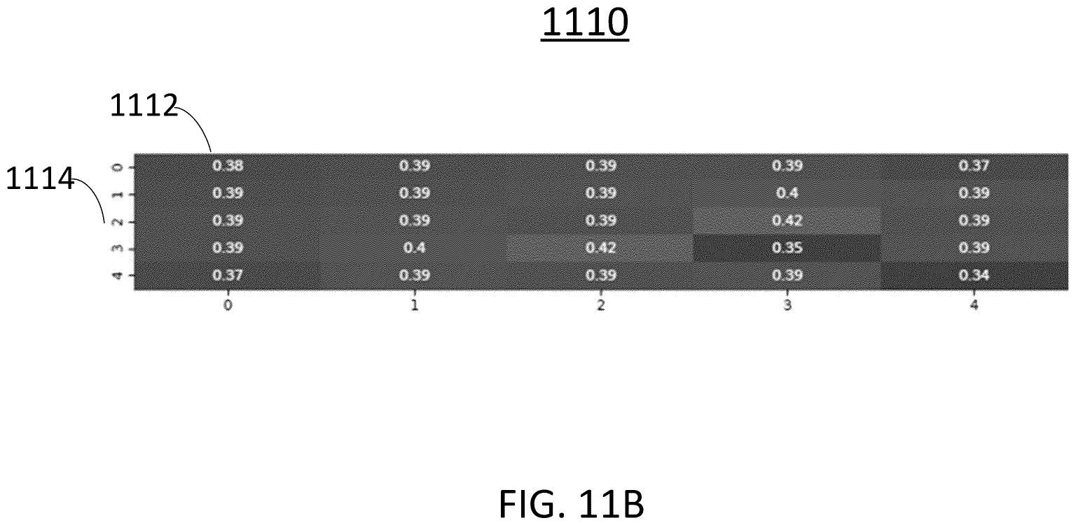

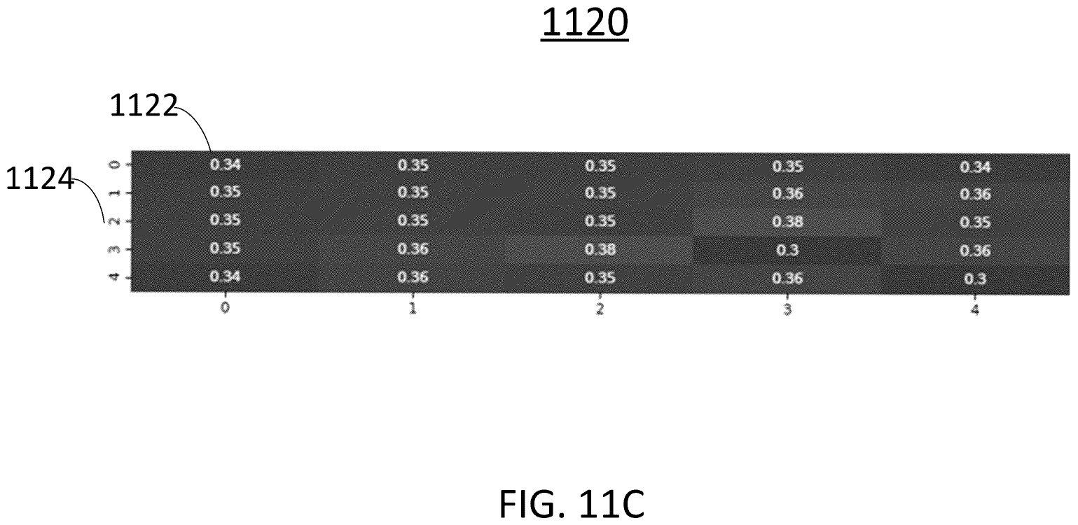

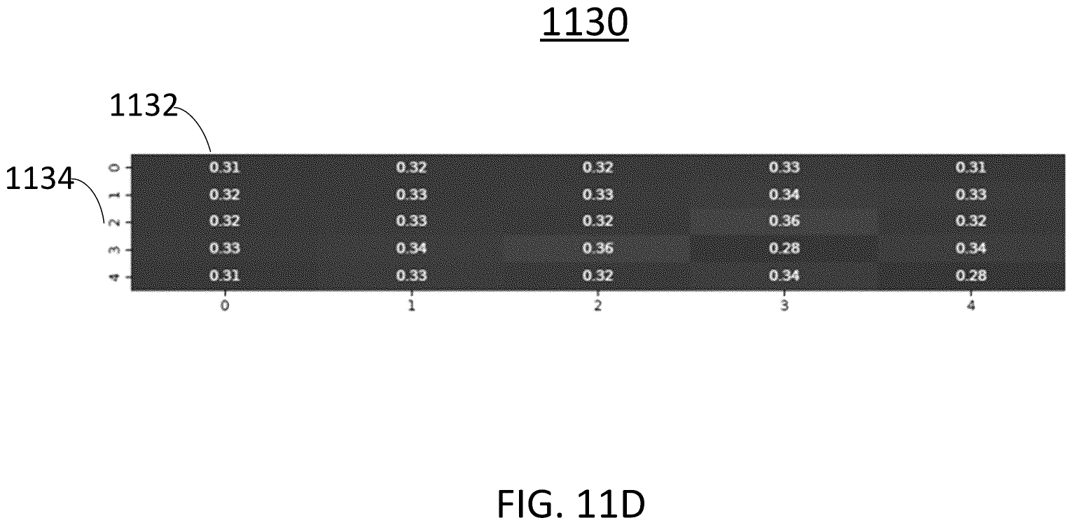

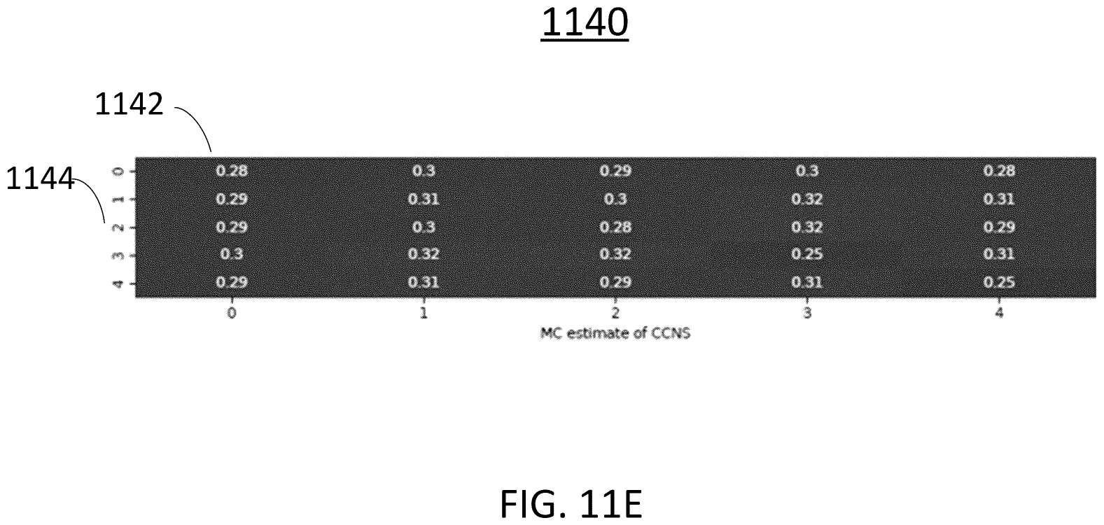

FIGS. 11A-E illustrates histograms of the number of layers chosen by each model on the different datasets during a 10-fold cross validation for risk assessment, in accordance with embodiments of the present disclosure.

FIG. 11A depicts a table 1100 that represents a true cross-class neighborhood similarity, when k is selected as 5.

FIG. 11B depicts a table 11100 that represents a true cross-class neighborhood similarity, when k is selected as 25.

FIG. 11C depicts a table 1120 that represents a true cross-class neighborhood similarity, when k is selected as 50.

FIG. 11D depicts a table 1130 that represents a true cross-class neighborhood similarity, when k is selected as 100.

FIG. 11E depicts a table 1140 that represents data related to a Monte Carlo simulation estimate of cross-class neighborhood similarity.

From tables 11A to 11E, it can be understood that the true cross-class neighborhood similarity, as depicted in tables 11A-11D, gets closer to the Monte Carlo approximation when the number of neighbors k is increased. The choice of the F parameter for the MC estimation of the cross-class neighborhood similarity has a marginal impact here. This behavior is expected, because, as k is increased a better approximation of the posterior class distribution in the hypercube around each point is obtained, which is the one computed previously. When k is too small, the empirical (true) cross-class neighbor similarity might be more unstable.

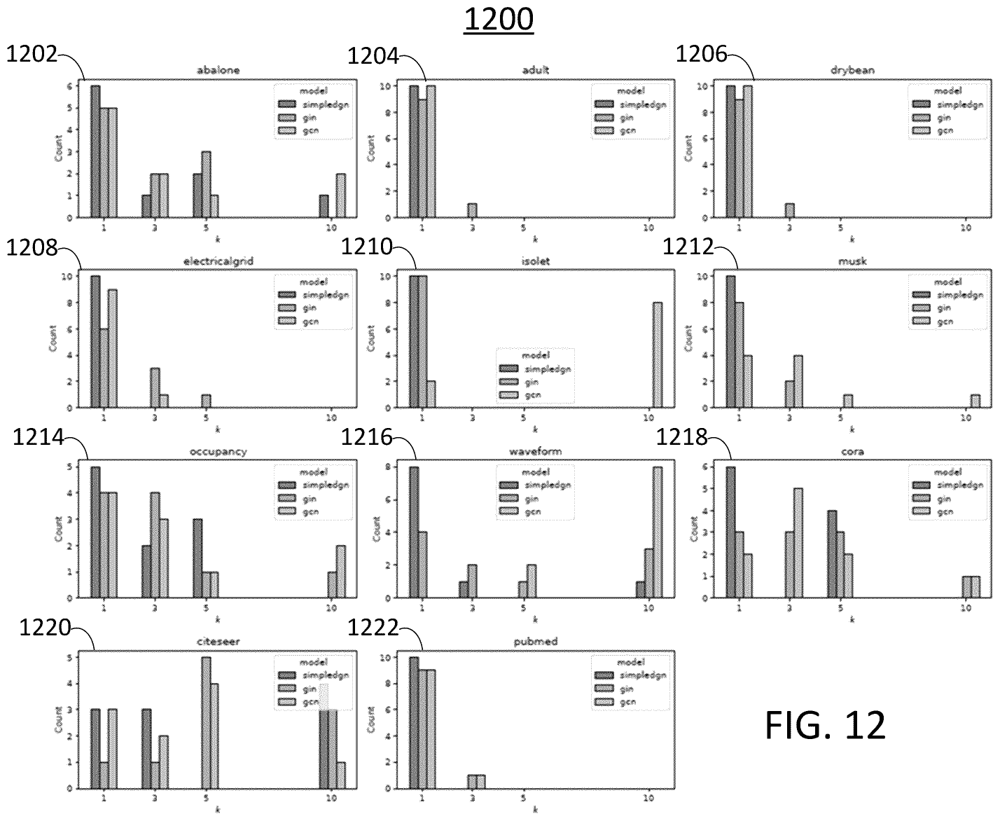

FIGS. 12-14 depict the histograms for each dataset of the 11 datasets when using a 10-fold cross validation for risk assessment. For example, each histogram in FIGS. 12-14 depicts a count (plotted on the y-axis) of a hyper-parameter k, number of layers, or number of hidden units (plotted on the x-axis) chosen by each family of model on different datasets. The counts on the y-axis shows how many configurations with a specific hyper-parameter value were selected as “best” across the different datasets. Each histogram also includes a legend that identifies the various families of models that are plotted on each graph for each value on the x-axis. For example, the legend in each graph can identify a subset of four families of models plotted in each graph. The four families of identified models include the simple deep graph network, the graph convolutional network, the graph isomorphism network, and MLP. In machine learning, the model selection phase selects the best hyper-parameters of a model (number of layers, number of hidden neurons etc.) that maximize performance on a “validation set”. If many different combinations are tried, some of them will share a certain parameter, for instance the number of layers. Hence, if the hyper-parameters are filtered for number of layers=2, more than one of such configurations can be uncovered.

FIG. 12 depicts histogram 1202 which is a plot of a count (on the y-axis) of k-values (on the x-axis) chosen by each of the three families of models (simple deep graph network, the graph convolutional network, and the graph isomorphism network) based on data stored in dataset ABALONE, histogram 1204 which is a plot of a count (on the y-axis) of k-values (on the x-axis) chosen by the three families of models based on data stored in dataset ADULT, histogram 1206 which is a plot of a count (on the y-axis) of k-values (on the x-axis) chosen by the three families of models based on data stored in dataset DRY BEAN, histogram 1208 which is a plot of a count (on the y-axis) of k-values (on the x-axis) chosen by the three families of models based on data stored in dataset ELECTRICAL GRID STABILITY, histogram 1210 which is a plot of a count (on the y-axis) of k-values (on the x-axis) chosen by the three families of models based on data stored in dataset ISOLET, histogram 1212 which is a plot of a count (on the y-axis) of k-values (on the x-axis) chosen by the three families of models based on data stored in dataset MUSK, histogram 1214 which is a plot of a count (on the y-axis) of k-values (on the x-axis) chosen by the three families of models based on data stored in dataset OCCUPANCY DETECTION, histogram 1216 which is a plot of a count (on the y-axis) of k-values (on the x-axis) chosen by the three families of models based on data stored in dataset WAVEFORM DATABASE GENERATOR, histogram 1218 which is a plot of a count (on the y-axis) of k-values (on the x-axis) chosen by the three families of models based on data stored in dataset CORA, histogram 1220 which is a plot of a count (on the y-axis) of k-values (on the x-axis) chosen by the three families of models based on data stored in dataset CITESEER, and histogram 1222 which is a plot a count (on the y-axis) of k-values (on the x-axis) chosen by the three families of models based on data stored in dataset PUBMED.

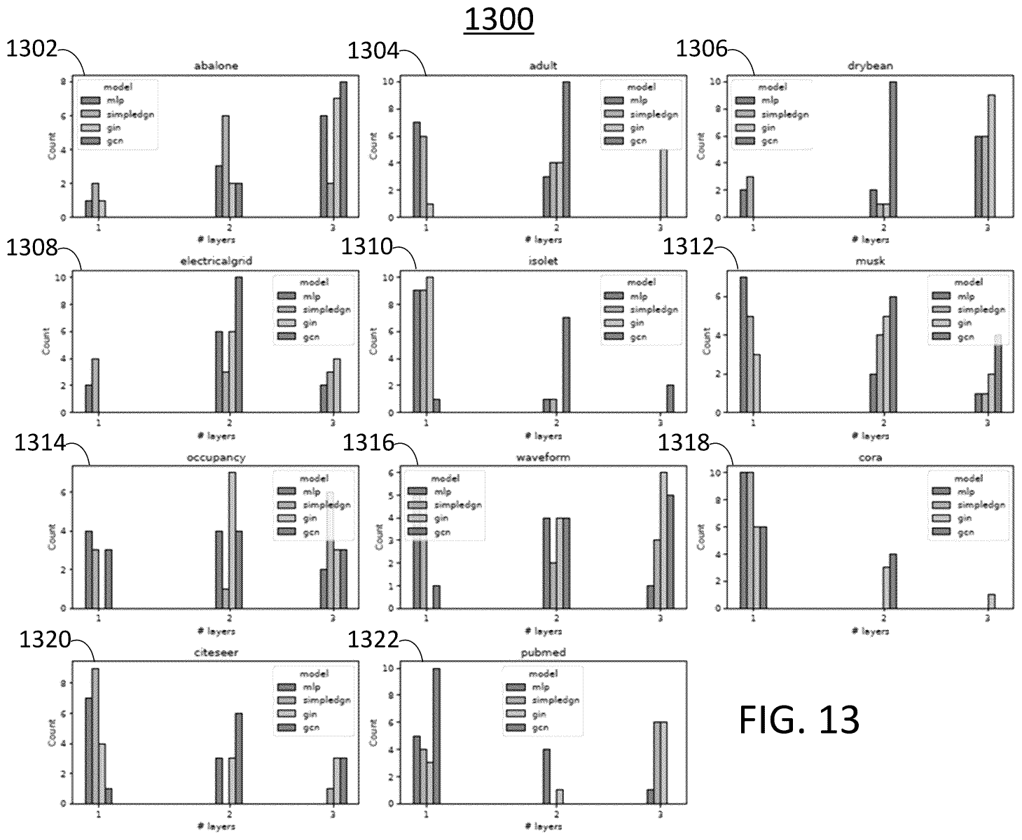

FIG. 13 depicts histogram 1302 which is a plot of count (on the y-axis) of number of layers (on the x-axis) chosen by the four families of models (simple deep graph network, the graph convolutional network, the graph isomorphism network, and MLP) based on data stored in dataset ABALONE, histogram 1304 which is a plot of count (on the y-axis) of number of layers (on the x-axis) chosen by the four families of models based on data stored in dataset ADULT, histogram 1306 which is a plot of count (on the y-axis) of number of layers (on the x-axis) chosen by the four families of models based on data stored in dataset DRY BEAN, histogram 1308 which is a plot of count (on the y-axis) of number of layers (on the x-axis) chosen by the four families of models based on data stored in dataset ELECTRICAL GRID STABILITY, histogram 1310 which is a plot of count (on the y-axis) of number of layers (on the x-axis) chosen by the four families of models based on data stored in dataset ISOLET, histogram 1312 which is a plot of count (on the y-axis) of number of layers (on the x-axis) chosen by the four families of models based on data stored in dataset MUSK, histogram 1314 which is a plot of count (on the y-axis) of number of layers (on the x-axis) chosen by the four families of models based on data stored in dataset OCCUPANCY DETECTION, histogram 1316 which is a plot of count (on the y-axis) of number of layers (on the x-axis) chosen by the four families of models based on data stored in dataset WAVEFORM DATABASE GENERATOR, histogram 1318 which is a plot of count (on the y-axis) of number of layers (on the x-axis) chosen by the four families of models based on data stored in dataset CORA, histogram 1320 which is a plot of count (on the y-axis) of number of layers (on the x-axis) chosen by the four families of models based on data stored in dataset CITESEER, and histogram 1322 which is a plot of count (on the y-axis) of number of layers (on the x-axis) chosen by the four families of models based on data stored in dataset PUBMED.

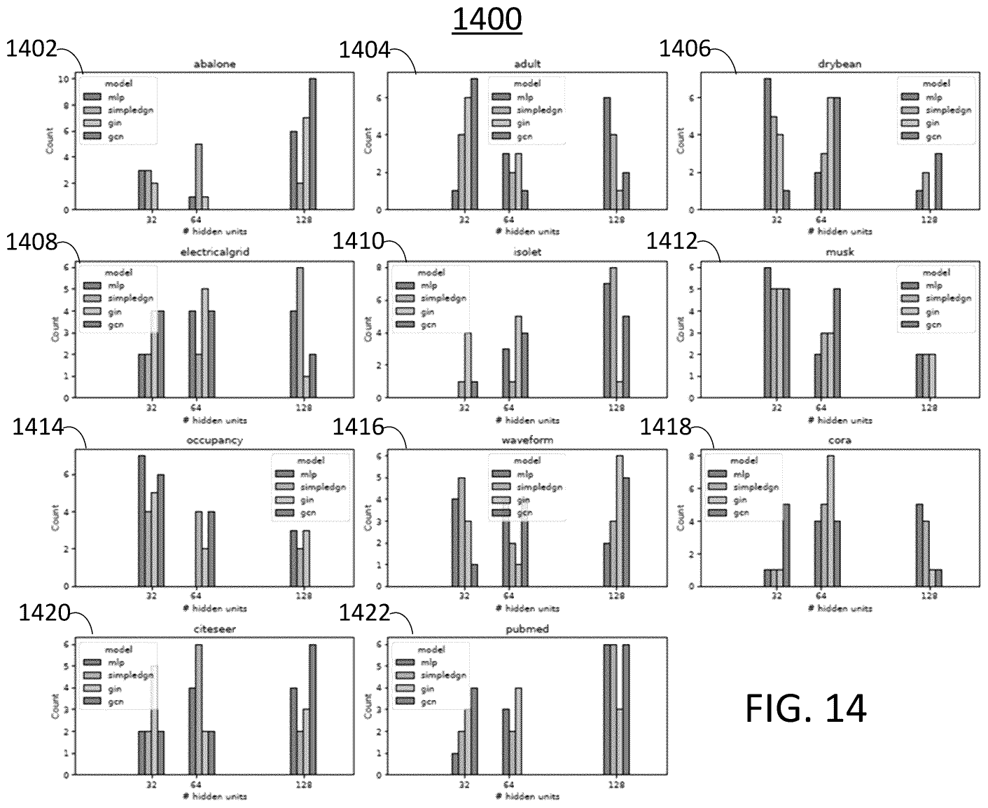

FIG. 14 depicts histogram 1402 which is a plot of count (on the y-axis) of number of hidden units (on the x-axis) chosen by the four families of models (simple deep graph network, the graph convolutional network, the graph isomorphism network, and MLP) based on data stored in dataset ABALONE, histogram 1404 which is a plot of count (on the y-axis) of number of hidden units (on the x-axis) chosen by the four families of models based on data stored in dataset ADULT, histogram 1406 which is a plot of count (on the y-axis) of number of hidden units (on the x-axis) chosen by the four families of models based on data stored in dataset DRY BEAN, histogram 1408 which is a plot of count (on the y-axis) of number of hidden units (on the x-axis) chosen by the four families of models based on data stored in dataset ELECTRICAL GRID STABILITY, histogram 1410 which is a plot of count (on the y-axis) of number of hidden units (on the x-axis) chosen by the four families of models based on data stored in dataset ISOLET, histogram 1412 which is a plot of count (on the y-axis) of number of hidden units (on the x-axis) chosen by the four families of models based on data stored in dataset MUSK, histogram 1414 which is a plot of count (on the y-axis) of number of hidden units (on the x-axis) chosen by the four families of models based on data stored in dataset OCCUPANCY DETECTION, histogram 1416 which is a plot of count (on the y-axis) of number of hidden units (on the x-axis) chosen by the four families of models based on data stored in dataset WAVEFORM DATABASE GENERATOR, histogram 1418 which is a plot of count (on the y-axis) of number of hidden units (on the x-axis) chosen by the four families of models based on data stored in dataset CORA, histogram 1420 which is a plot of count (on the y-axis) of number of hidden units (on the x-axis) chosen by the four families of models based on data stored in dataset CITESEER, and histogram 1422 which is a plot of count (on the y-axis) of number of hidden units (on the x-axis) chosen by the four families of models based on data stored in dataset PUBMED.

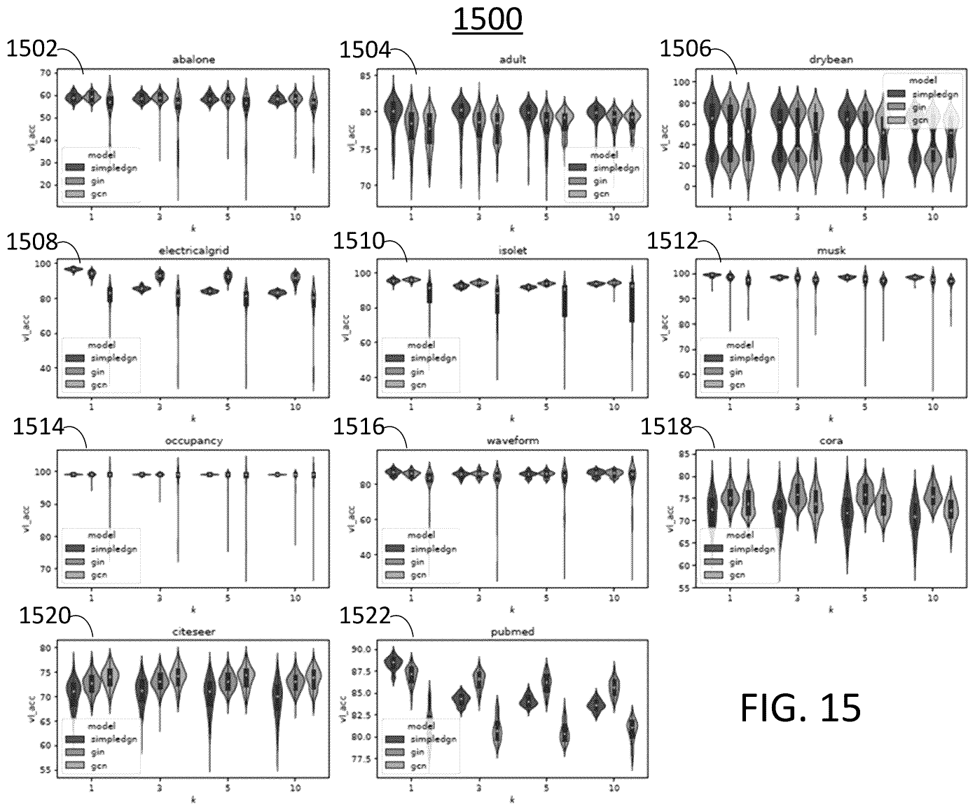

FIGS. 15-17 depicts ablation studies for the chosen k value, number of layers, number of hidden units, by each family of model on the different datasets. For example, each graph in FIGS. 15-17 depicts vl_acc on the y-axis and a hyper-parameter k, number of layers, or number of hidden units on the x-axis. In some embodiments, vl_acc refers to validation accuracy. Each plot reports the average and deviation for performance of the models with that specific value of the hyper-parameters (e.g., number of layers=2). Each graph also includes a legend that identifies the families of models that are plotted on each graph for each value on the x-axis. For example, the legend in each graph identifies a subset of four families of models plotted in each graph. The four families of identified models include the simple deep graph network, the graph convolutional network, the graph isomorphism network, and MLP. In machine learning, the model selection phase selects the best hyper-parameters of a model (number of layers, number of hidden neurons etc.) that maximize performance on a “validation set”. If many different combinations are tried, some of them will share a certain parameter, for instance the number of layers. Hence, if the hyper-parameters are filtered for number of layers=2, more than one of such configurations can be uncovered.

FIG. 15 depicts an ablation study 1502 which is a plot of vl_acc (on the y-axis) based on k-values (on the x-axis) chosen by each of the three families of models (simple deep graph network, the graph convolutional network, and the graph isomorphism network) based on data stored in dataset ABALONE, ablation study 1504 which is a plot of vl_acc (on the y-axis) based on k-values (on the x-axis) chosen by the three families of models based on data stored in dataset ADULT, ablation study 1506 which is a plot of vl_acc (on the y-axis) based on k-values (on the x-axis) chosen by the three families of models based on data stored in dataset DRY BEAN, ablation study 1508 which is a plot of vl_acc (on the y-axis) based on k-values (on the x-axis) chosen by the three families of models based on data stored in dataset ELECTRICAL GRID STABILITY, ablation study 1510 which is a plot of vl_acc (on the y-axis) based on k-values (on the x-axis) chosen by the three families of models based on data stored in dataset ISOLET, ablation study 1512 which is a plot of vl_acc (on the y-axis) based on k-values (on the x-axis) chosen by the three families of models based on data stored in dataset MUSK, ablation study 1514 which is a plot of vl_acc (on the y-axis) based on k-values (on the x-axis) chosen by the three families of models based on data stored in dataset OCCUPANCY DETECTION, ablation study 1516 which is a plot of vl_acc (on the y-axis) based on k-values (on the x-axis) chosen by the three families of models based on data stored in dataset WAVEFORM DATABASE GENERATOR, ablation study 1518 which is a plot of vl_acc (on the y-axis) based on k-values (on the x-axis) chosen by the three families of models based on data stored in dataset CORA, ablation study 1520 which is a plot of vl_acc (on the y-axis) based on k-values (on the x-axis) chosen by the three families of models based on data stored in dataset CITESEER, and ablation study 1222 which is a plot of vl_acc (on the y-axis) based on k-values (on the x-axis) chosen by the three families of models based on data stored in dataset PUBMED.

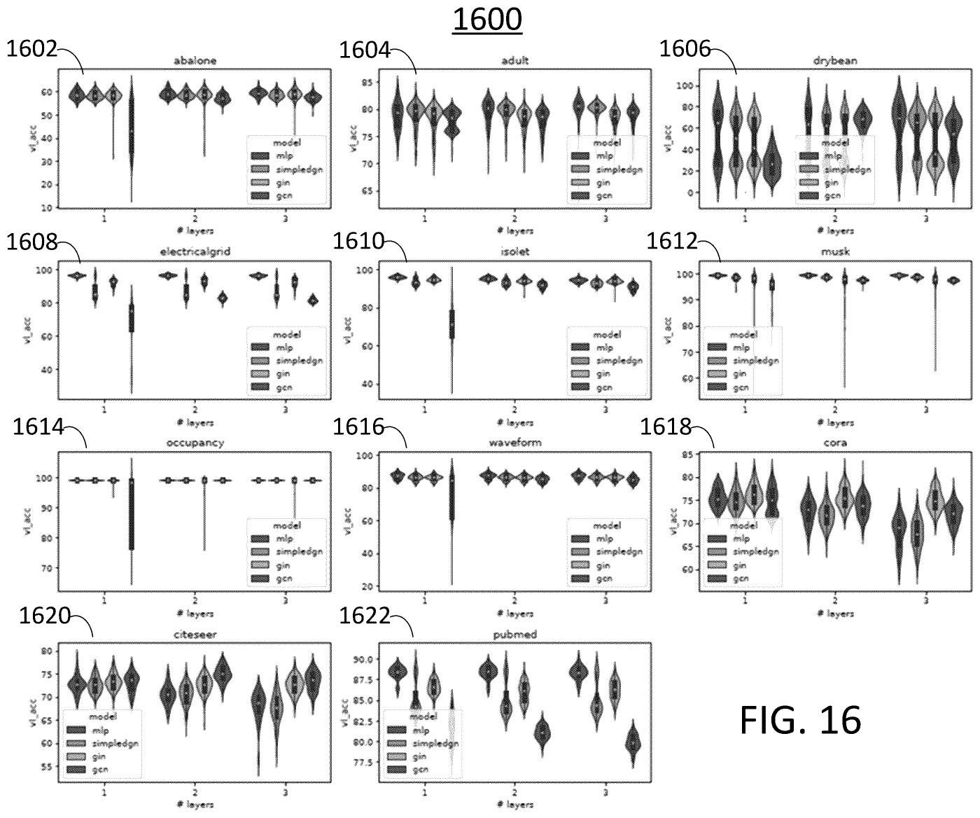

FIG. 16 depicts ablation study 1602 which is a plot of vl_acc (on the y-axis) based on the number of layers (on the x-axis) chosen by the four families of models (simple deep graph network, the graph convolutional network, the graph isomorphism network, and MLP) based on data stored in dataset ABALONE, ablation study 1604 which is a plot of vl_acc (on the y-axis) based on the number of layers (on the x-axis) chosen by the four families of models based on data stored in dataset ADULT, ablation study 1606 which is a plot of vl_acc (on the y-axis) based on the number of layers (on the x-axis) chosen by the four families of models based on data stored in dataset DRY BEAN, ablation study 1608 which is a plot of vl_acc (on the y-axis) based on the number of layers (on the x-axis) chosen by the four families of models based on data stored in dataset ELECTRICAL GRID STABILITY, ablation study 1610 which is a plot of vl_acc (on the y-axis) based on the number of layers (on the x-axis) chosen by the four families of models based on data stored in dataset ISOLET, ablation study 1612 which is a plot of vl_acc (on the y-axis) based on the number of layers (on the x-axis) chosen by the four families of models based on data stored in dataset MUSK, ablation study 1614 which is a plot of vl_acc (on the y-axis) based on the number of layers (on the x-axis) chosen by the four families of models based on data stored in dataset OCCUPANCY DETECTION, ablation study 1616 which is a plot of vl_acc (on the y-axis) based on the number of layers (on the x-axis) chosen by the four families of models based on data stored in dataset WAVEFORM DATABASE GENERATOR, ablation study 1618 which is a plot of vl_acc (on the y-axis) based on the number of layers (on the x-axis) chosen by the four families of models based on data stored in dataset CORA, ablation study 1620 which is a plot of vl_acc (on the y-axis) based on the number of layers (on the x-axis) chosen by the four families of models based on data stored in dataset CITESEER, and ablation study 1622 which is a plot of vl_acc (on the y-axis) based on the number of layers (on the x-axis) chosen by the four families of models based on data stored in dataset PUBMED.

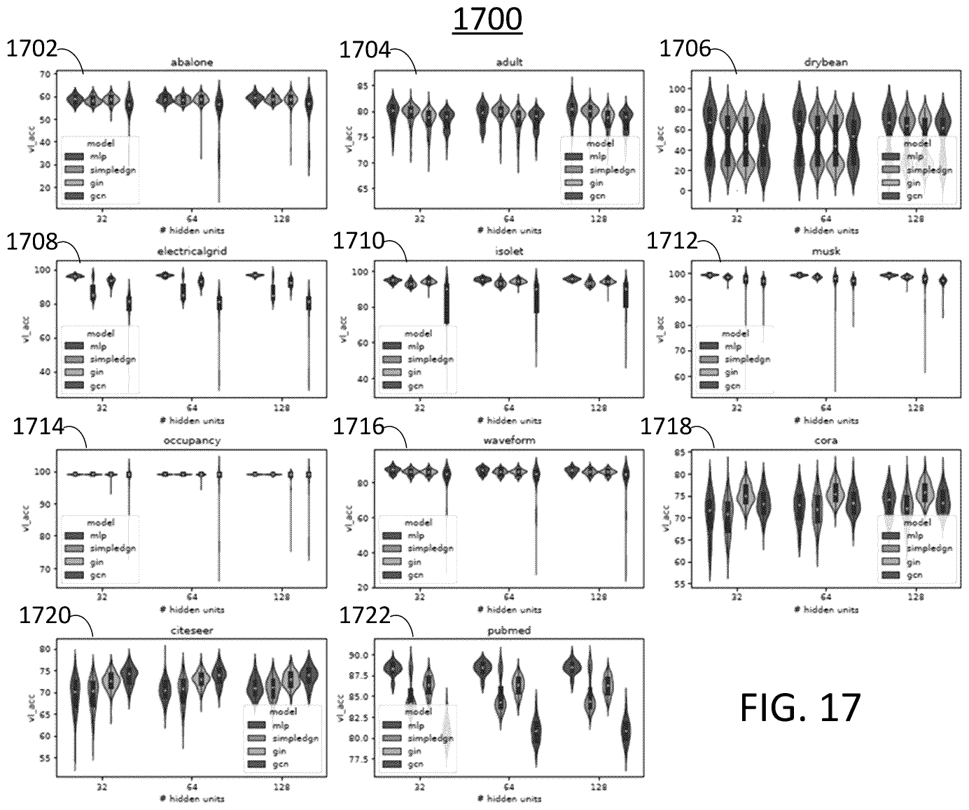

FIG. 17 depicts ablation study 1702 which is a plot of vl_acc (on the y-axis) based on the number of hidden units (on the x-axis) chosen by the four families of models (simple deep graph network, the graph convolutional network, the graph isomorphism network, and MLP) based on data stored in dataset ABALONE, ablation study 1704 which is a plot of vl_acc (on the y-axis) based on the number of hidden units (on the x-axis) chosen by the four families of models based on data stored in dataset ADULT, ablation study 1706 which is a plot of vl_acc (on the y-axis) based on the number of hidden units (on the x-axis) chosen by the four families of models based on data stored in dataset DRY BEAN, ablation study 1708 which is a plot of vl_acc (on the y-axis) based on the number of hidden units (on the x-axis) chosen by the four families of models based on data stored in dataset ELECTRICAL GRID STABILITY, ablation study 1710 which is a plot of vl_acc (on the y-axis) based on the number of hidden units (on the x-axis) chosen by the four families of models based on data stored in dataset ISOLET, ablation study 1712 which is a plot of vl_acc (on the y-axis) based on the number of hidden units (on the x-axis) chosen by the four families of models based on data stored in dataset MUSK, ablation study 1714 which is a plot of vl_acc (on the y-axis) based on the number of hidden units (on the x-axis) chosen by the four families of models based on data stored in dataset OCCUPANCY DETECTION, ablation study 1716 which is a plot of vl_acc (on the y-axis) based on the number of hidden units (on the x-axis) chosen by the four families of models based on data stored in dataset WAVEFORM DATABASE GENERATOR, ablation study 1718 which is a plot of vl_acc (on the y-axis) based on the number of hidden units (on the x-axis) chosen by the four families of models based on data stored in dataset CORA, ablation study 1720 which is a plot of vl_acc (on the y-axis) based on the number of hidden units (on the x-axis) chosen by the four families of models based on data stored in dataset CITESEER, and ablation study 1722 which is a plot of vl_acc (on the y-axis) based on the number of hidden units (on the x-axis) chosen by the four families of models based on data stored in dataset PUBMED.

The following list of references is hereby incorporated by reference herein:
Uri Alon and Eran Yahav. On the bottleneck of graph neural networks and its practical implications. In 9th International Conference on Learning Representations (ICLR), 2021.
Arthur Asuncion and David Newman. Uci machine learning repository, 2007. URL https://archive.ics.uci.edu/ml/index.php.
Davide Bacciu, Federico Errica, and Alessio Micheli. Probabilistic learning on graphs via contextual architectures. Journal of Machine Learning Research, 21(134):1-39, 2020.
Davide Bacciu, Federico Errica, Alessio Micheli, and Marco Podda. A gentle introduction to deep learning for graphs. Neural Networks, 129:203-221, 9 2020.
Aseem Baranwal, Kimon Fountoulakis, and Aukosh Jagannath. Graph convolution for semi-supervised classification: Improved linear separability and out-of-distribution generalization. In Proceedings of the 38^{th} International Conference on Machine Learning (ICML), 2021.
Adrien Benamira, Benjamin Devillers, Etienne Lesot, Ayush K Ray, Manal Saadi, and Fragkiskos D Malliaros. Semi-supervised learning and graph neural networks for fake news detection. In Proceedings of the IEEE/ACM International Conference on Advances in SocialNetworks Analysis and Mining (ASONAM), 2019.
Cristian Bodnar, Fabrizio Frasca, Nina Otter, Yuguang Wang, Pietro Lio, Guido F Montufar, and Michael Bronstein. Weisfeiler and lehman go cellular: Cw networks. In Proceedings of the 35^{th} Conference on Neural Information Processing Systems (NeurIPS), 2021.
Cristian Bodnar, Francesco Di Giovanni, Benjamin Paul Chamberlain, Pietro Lio, and Michael M. Bronstein. Neural sheaf diffusion: A topological perspective on heterophily and oversmoothing in GNNs. In Proceedings of the 36^{th} Conference on Neural Information Processing Systems (NeurIPS), 2022.
Michael M. Bronstein, Joan Bruna, Yann LeCun, Arthur Szlam, and Pierre Vandergheynst. Geometric deep learning: going beyond Euclidean data. IEEE Signal Processing Magazine, 34(4):18-42, 2017.
Andrea Cavallo, Claas Grohnfeldt, Michele Russo, Giulio Lovisotto, and Luca Vassio. 2-hop neighbor class similarity (2ncs): A graph structural metric indicative of graph neural network performance. Workshop on Graphs and more Complex structures for Learning and Reasoning (AAAI), 2023.
Ben Chamberlain, James Rowbottom, Maria I Gorinova, Michael Bronstein, Stefan Webb, and Emanuele Rossi. Grand: Graph neural diffusion. In Proceedings of the 38^{th} International Conference on Machine Learning (ICML). PMLR, 2021.
Deli Chen, Yankai Lin, Wei Li, Peng Li, Jie Zhou, and Xᵤ Sun. Measuring and relieving the over-smoothing problem for graph neural networks from the topological view. In Proceedings of the 34^{th} AAAI Conference on Artificial Intelligence (AAAI), pages 3438-3445, 2020.
Ming Chen, Zhewei Wei, Zengfeng Huang, Bolin Ding, and Yaliang Li. Simple and deep graph convolutional networks. In Proceedings of the 37^{th} International Conference on Machine Learning (ICML), 2020.
Yu Chen, Lingfei Wu, and Mohammed Zaki. Iterative deep graph learning for graph neural networks: Better and robust node embeddings. In Proceedings of the 34^{th} Conference on Neural Information Processing Systems (NeurIPS), 2020.
Pedro Domingos. A few useful things to know about machine learning. Communications of the ACM, 55(10):78-87, 2012.
Vijay Prakash Dwivedi and Xavier Bresson. A generalization of transformer networks to graphs. Workshop on Deep Learning on Graphs: Methods and Applications (AAAI), 2021.
David Eppstein, Michael S Paterson, and F Frances Yao. On nearest-neighbor graphs. Discrete & Computational Geometry, 17:263-282, 1997.
Federico Errica, Marco Podda, Davide Bacciu, and Alessio Micheli. A fair comparison of graph neural networks for graph classification. In 8^{th} International Conference on Learning Representations (ICLR), 2020.
Bahare Fatemi, Layla El Asri, and Seyed Mehran Kazemi. Slaps: Self-supervision improves structure learning for graph neural networks. In Proceedings of the 35^{th} Conference on Neural Information Processing Systems (NeurIPS), 2021.
Luca Franceschi, Mathias Niepert, Massimiliano Pontil, and Xiao He. Learning discrete structures for graph neural networks. In Proceedings of the 36^{th} International Conference on Machine Learning (ICML), 2019.
Paolo Frasconi, Marco Gori, and Alessandro Sperduti. A general framework for adaptive processing of data structures. IEEE Transactions on Neural Networks, 9(5):768-786, 1998.
Floris Geerts, Filip Mazowiecki, and Guillermo Perez. Let's agree to degree: Comparing graph convolutional networks in the message-passing framework. In Proceedings of the 38^{th} International Conference on Machine Learning (ICML), 2021.
Justin Gilmer, Samuel S Schoenholz, Patrick F Riley, Oriol Vinyals, and George E Dahl. Neural message passing for quantum chemistry. In Proceedings of the 34^{th} International Conference on Machine Learning (ICML), 2017.
Alessio Gravina, Davide Bacciu, and Claudio Gallicchio. Anti-symmetric dgn: a stable architecture for deep graph networks. In 11^{th} International Conference on Learning Representations (ICLR), 2023.
Will Hamilton, Zhitao Ying, and Jure Leskovec. Inductive representation learning on large graphs. In Proceedings of the 31^{st} Conference on Neural Information Processing Systems (NIPS), 2017.
William L. Hamilton, Rex Ying, and Jure Leskovec. Representation learning on graphs: Methods and applications. IEEE Data Engineering Bulletin, 40(3):52-74, 2017.
Marko Helén and Tuomas Virtanen. Query by example of audio signals using euclidean distance between gaussian mixture models. In Proceedings of the IEEE International Conference on Acoustics, Speech and Signal Processing (ICASSP), 2007.
Anees Kazi, Luca Cosmo, Seyed-Ahmad Ahmadi, Nassir Navab, and Michael M Bronstein. Differentiable graph module (dgm) for graph convolutional networks. IEEE Transactions on Pattern Analysis and Machine Intelligence, 45(2):1606-1617, 2022.
Diederik P. Kingma and Jimmy Ba. Adam: A method for stochastic optimization. In 3^{rd} International Conference on Learning Representations (ICLR), 2015.
Thomas N Kipf and Max Welling. Semi-supervised classification with graph convolutional networks. In 5^{th} International Conference on Learning Representations (ICLR), 2017.
Devin Kreuzer, Dominique Beaini, Will Hamilton, Vincent Létourneau, and Prudencio Tossou. Rethinking graph transformers with spectral attention. In Proceedings of the 35^{th} Conference on Neural Information Processing Systems (NeurIPS), 2021.
Nils M Kriege, Fredrik D Johansson, and Christopher Morris. A survey on graph kernels. Applied Network Science, 5(1):1-42, 2020.
Ao Li, Zhou Qin, Runshi Liu, Yiqun Yang, and Dong Li. Spam review detection with graph convolutional networks. In Proceedings of the 28th ACM International Conference on Information and Knowledge Management (CIKM), 2019.
Qimai Li, Zhichao Han, and Xiao-Ming Wu. Deeper insights into graph convolutional networks for semi-supervised learning. In Proceedings of the 32nd AAAI Conference on Artificial Intelligence (AAAI), 2018.
Yi-Lun Liao and Tess Smidt. Equiformer: Equivariant graph attention transformer for 3d atomistic graphs. In 11th International Conference on Learning Representations (ICLR), 2023.
Siyuan Lu, Ziquan Zhu, Juan Manuel Gorriz, Shui-Hua Wang, and Yu-Dong Zhang. Nagnn: classification of covid-19 based on neighboring aware representation from deep graph neural network. International Journal of Intelligent Systems, 37(2):1572-1598, 2022.
Yao Ma, Xiaorui Liu, Neil Shah, and Jiliang Tang. Is homophily a necessity for graph neural networks? In 10^{th} International Conference on Learning Representations (ICLR), 2022.
Brandon Malone, Alberto Garcia-Duran, and Mathias Niepert. Learning representations of missing data for predicting patient outcomes. In Workshop on Deep Learning on Graphs: Method and Applications (AAAI), 2021.
Miller McPherson, Lynn Smith-Lovin, and James M Cook. Birds of a feather: Homophily in social networks. Annual review of sociology, 27(1):415-444, 2001.
Alessio Micheli. Neural network for graphs: A contextual constructive approach. IEEE Transactions on Neural Networks, 20(3):498-511, 2009.
Christopher Morris, Martin Ritzert, Matthias Fey, William L Hamilton, Jan Eric Lenssen, Gaurav Rattan, and Martin Grohe. Weisfeiler and leman go neural: Higher-order graph neural networks. In Proceedings of the 33rd AAAI Conference on Artificial Intelligence (AAAI), volume 33, 2019.
Luis Müller, Mikhail Galkin, Christopher Morris, and Ladislav Rampasek. Attending to graph transformers. arXiv preprint arXiv:2302.04181, 2023.
Galileo Mark Namata, Ben London, Lise Getoor, and Bert Huang. Query-driven active surveying for collective classification. In Proceedings of the Workshop on Mining and Learning with Graphs (MLG), 2012.
Mathias Niepert, Mohamed Ahmed, and Konstantin Kutzkov. Learning convolutional neural networks for graphs. In Proceedings of the 33rd International Conference on Machine Learning (ICML), 2016.
K B Petersen and MS Pedersen. The matrix cookbook, version 20121115. Technical report, Technical University of Denmark, 2012.
Oleg Platonov, Denis Kuznedelev, Michael Diskin, Artem Babenko, and Liudmila Prokhorenkova. A critical look at the evaluation of GNNs under heterophily: Are we really making progress? In 11^{th} International Conference on Learning Representations (ICLR), 2023.
Lutz Prechelt. Early stopping—but when? Neural networks: tricks of the trade: second edition, pages 53-67, 2012.
Rahul Ragesh, Sundararajan Sellamanickam, Arun Iyer, Ramakrishna Bairi, and Vijay Lingam. Hetegcn: heterogeneous graph convolutional networks for text classification. In Proceedings of the 14^{th} ACM International Conference on Web Search and Data Mining (WSDM), 2021.
Frank Rosenblatt. The perceptron: a probabilistic model for information storage and organization in the brain. Psychological review, 65(6):386, 1958.
Franco Scarselli, Marco Gori, Ah Chung Tsoi, Markus Hagenbuchner, and Gabriele Monfardini. The graph neural network model. IEEE Transactions on Neural Networks, 20(1):61-80, 2009.
Oliver C Schrempf, Olga Feiermann, and Uwe D Hanebeck. Optimal mixture approximation of the product of mixtures. In 7^{th} International Conference on Information Fusion (FUSION), 2005.
Prithviraj Sen, Galileo Namata, Mustafa Bilgic, Lise Getoor, Brian Galligher, and Tina Eliassi Rad. Collective classification in network data. AI magazine, 29(3):93-93, 2008.
Oleksandr Shchur, Maximilian Mumme, Aleksandar Bojchevski, and Stephan Günnemann. Pitfalls of graph neural network evaluation. Workshop on Relational Representation Learning, Neural Information Processing Systems (NeurIPS), 2018.
Alessandro Sperduti and Antonina Starita. Supervised neural networks for the classification of structures. IEEE Transactions on Neural Networks, 8(3):714-735, 1997.
Catherine Tong, Emma Rocheteau, Petar Velic ̆kovic', Nicholas Lane, and Pietro Liò. Predicting patient outcomes with graph representation learning. In AI for Disease Surveillance and Pandemic Intelligence: Intelligent Disease Detection in Action, pages 281-293. Springer, 2022.
Jake Topping, Francesco Di Giovanni, Benjamin Paul Chamberlain, Xiaowen Dong, and Michael M Bronstein. Understanding over-squashing and bottlenecks on graphs via curvature. In 10^{th} International Conference on Learning Representations (ICLR), 2022.
Domenico Tortorella and Alessio Micheli. Leave graphs alone: Addressing over-squashing without rewiring. In Proceedings of the 1st Conference on Learning on Graphs (LOG), 2022.
Petar Velickovic, Guillem Cucurull, Arantxa Casanova, Adriana Romero, Pietro Lio, and Yoshua Bengio. Graph attention networks. In 6^{th} International Conference on Learning Representations (ICLR), 2018.
S Vichy N Vishwanathan, Nicol N Schraudolph, Risi Kondor, and Karsten M Borgwardt. Graph kernels. Journal of Machine Learning Research, 11(April):1201-1242, 2010.
Yue Wang, Yongbin Sun, Ziwei Liu, Sanjay E Sarma, Michael M Bronstein, and Justin M Solomon. Dynamic graph cnn for learning on point clouds. Acm Transactions On Graphics (tog), 38(5):1-12, 2019.
Zonghan Wu, Shirui Pan, Fengwen Chen, Guodong Long, Chengqi Zhang, and S Yu Philip. A comprehensive survey on graph neural networks. IEEE Transactions on Neural Networks and Learning Systems, 32(1):4-24, 2020.
Keyulu Xu, Weihua Hu, Jure Leskovec, and Stefanie Jegelka. How powerful are graph neural networks? In 7^{th} International Conference on Learning Representations (ICLR), 2019.
Chengxuan Ying, Tianle Cai, Shengjie Luo, Shuxin Zheng, Guolin Ke, Di He, Yanming Shen, and Tie-Yan Liu. Do transformers really perform badly for graph representation? In Proceedings of the 35^{th} Conference on Neural Information Processing Systems (NeurIPS), 2021.
Donghan Yu, Ruohong Zhang, Zhengbao Jiang, Yuexin Wu, and Yiming Yang. Graph-revised convolutional network. In Proceedings of the European Conference on Machine Learning and Principles and Practice of Knowledge Discovery in Databases (ECML PKDD), 2021.
Xiang Yu, Siyuan Lu, Lili Guo, Shui-Hua Wang, and Yu-Dong Zhang. Resgnet-c: A graph convolutional neural network for detection of covid-19. Neurocomputing, 452:592-605, 2021.
Ziwei Zhang, Peng Cui, and Wenwu Zhu. Deep learning on graphs: A survey. IEEE Transactions on Knowledge and Data Engineering, 34(1):249-270, 2020.
Jiong Zhu, Yujun Yan, Lingxiao Zhao, Mark Heimann, Leman Akoglu, and Danai Koutra. Beyond homophily in graph neural networks: Current limitations and effective designs. Proceedings of the 34^{th} Conference on Neural Information Processing Systems (NeurIPS), 2020.

Embodiments of the present invention can be advantageously applied to regression problems (continuous values) to provide improvements to various technical fields such as operation system design and optimization, material design and optimization, telecommunication network design and optimization, etc. Compared to existing approaches, embodiments of the present invention minimize uncertainty, while increasing performance and accuracy, providing for faster computation and saving computational resources and memory. For example, according to embodiments of the present invention, outliers with low uncertainty can be avoided while the latency and/or memory consumption is linear or constant.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications can be made, by those of ordinary skill in the art, within the scope of the following claims, which can include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A method for construction of nearest neighbor structures, the method comprising:
determining a set of cross-class neighborhood similarities based on a set of distributions of data obtained by applying a model to data present in a dataset;
selecting a first cross-class neighborhood similarity from the set of cross-class neighborhood similarities based on one or more inter-class cross-class neighborhood similarities and one or more intra-class cross-class neighborhood similarities; and
building a nearest neighbor graph based on the first cross-class neighborhood similarity.

2. The method of claim 1, applying the model to data present in the dataset comprises:
receiving data for the dataset in a tabular from via user input; and
modeling each data point of the data that belongs to a class (C) as a mixture of Gaussian distributions (M).

3. The method of claim 2, wherein applying the model to data present in the dataset further comprises determining learned parameters for the set of distributions of data present in the dataset.

4. The method of claim 3, wherein determining the set of cross-class neighborhood similarities comprises using the learned parameters to compute a value of the cross-class neighborhood similarities, wherein the nearest neighbor graph is built based on the value of the cross-class neighborhood similarities.

5. The method of claim 4, wherein the value of the cross-class neighborhood similarities is computed using Monte Carlo simulations.

6. The method of claim 1, further comprising:
training a graph machine learning model based on the nearest neighbor graph; and
performing predictive tasks using the trained graph machine learning model.

7. The method of claim 1, wherein the model applied to the data present in the dataset is a Hierarchical Naïve Bayes model, wherein the Hierarchical Naïve Bayes model models the set of distributions of the data as a mixture of Gaussian distributions, and wherein mixing weights of the mixture are obtained by another mixture of categorical distributions.

8. The method of claim 1, wherein determining the set of distributions comprises computing a probability that a first node, belonging to a first class, has a nearest neighbor node, belonging to a second class.

9. The method of claim 1, wherein selecting the first cross-class neighborhood similarity comprises determining a trade-off between the one or more inter-class cross-class neighborhood similarities and the one or more intra-class cross-class neighborhood similarities.

10. The method of claim 1, wherein the nearest neighbor graph comprises:
a selected node at a center of hypercube, wherein the hypercube comprises an edge that is optimized; and
a set of neighbors of the selected node within the hypercube based on the edge of the hypercube, wherein the hypercube is formed based on the first parameter.

11. The method of claim 1, wherein:
the data present in the dataset comprises electronic health records corresponding to a plurality of patients, wherein the electronic health records comprise heart rate, oxygen saturation, weight, height, glucose, temperature associated with each patient in the plurality of patients;
the nearest neighbor graph is built based on the electronic health records present in the dataset;
a graph machine learning model is trained using the nearest neighbor graph; and
a clinical risk is predicted for a patient using the trained graph machine learning model.

12. The method of claim 1, wherein:
the data present in the dataset comprises genomic activity information corresponding to a plurality of patients, wherein the genomic activity of each patient identifies a response of the respective patient to a drug;
the nearest neighbor graph is built based on the genomic activity information present in the dataset;
a graph machine learning model is trained using the nearest neighbor graph; and
a suitability of a patient for a drug trial is predicted using the graph machine learning model.

13. The method of claim 1, wherein:
the data present in the dataset comprises soil data corresponding to a plurality of areas, wherein the soil data comprises humidity, temperature, and performance metrics related to different areas;
the nearest neighbor graph is built based on the soil data present in the dataset;
a graph machine learning model is trained using the nearest neighbor graph; and
a quality of an input soil type is predicted based on the nearest neighbor graph.

14. A computer system programmed for performing automated sharing of data and analytics across a data space platform, the computer system comprising one or more hardware processors which, alone or in combination, are configured to provide for execution of the following steps:
determining a set of cross-class neighborhood similarities based on a set of distributions of data obtained by applying a model to data present in a dataset;
selecting a first cross-class neighborhood similarity from the set of cross-class neighborhood similarities based on one or more inter-class cross-class neighborhood similarities and one or more intra-class cross-class neighborhood similarities; and
building a nearest neighbor graph based on the first cross-class neighborhood similarity.

15. A tangible, non-transitory computer-readable medium for performing automated sharing of data and analytics across a data space platform having instructions thereon, which, upon being executed by one or more processors, provides for execution of the following steps:
determining a set of cross-class neighborhood similarities based on a set of distributions of data obtained by applying a model to data present in a dataset;
selecting a first cross-class neighborhood similarity from the set of cross-class neighborhood similarities based on one or more inter-class cross-class neighborhood similarities and one or more intra-class cross-class neighborhood similarities; and
building a nearest neighbor graph based on the first cross-class neighborhood similarity.
